(19) United States
(12) Patent Application Publication
(10) Pub. No.: US 2022/0348952 A1
(43) Pub. Date: Nov. 3, 2022

(54) COMPOSITIONS AND METHODS CONFERRING RESISTANCE TO RUST DISEASES

(71) Applicant: TECHNOLOGY INNOVATION MOMENTUM FUND (ISRAEL) LIMITED PARTNERSHIP, Tel-Aviv (IL)

(72) Inventors: Amir SHARON, Tel Aviv (IL); Eitan MILLET, Tel Aviv (IL); Anna MINZ-DUB, Tel Aviv (IL); Sofia KHAZAN, Tel Aviv (IL)

(21) Appl. No.: 17/624,002

(22) PCT Filed: Jul. 2, 2020
(86) PCT No.: PCT/IL2020/050739

§ 371 Date: Dec. 30, 2021

Related U.S. Application Data
(60) Provisional application No. 62/870,070, filed on Jul. 3, 2019.

(51) Int. Cl. C12N 15/82; C12Q 1/6895

(52) U.S. Cl. CPC C12N 15/8282; C12Q 1/6895; C12Q 2600/13

(57) ABSTRACT

The present invention relates to polynucleotides which confer or enhance resistance or tolerance to leaf rust and stripe rust disease onto wheat plants. The present invention further relates to methods of using the resistance-conferring polynucleotides for producing resistant or tolerant wheat plants and to heat plants so produced.

### FIELD OF THE INVENTION

The present invention relates to polynucleotides which confer or enhance resistance or tolerance of wheat plants towards the rust diseases leaf rust and stripe rust and to wheat plants comprising the polynucleotides that are resistant or tolerant to the rust disease as well as to methods of producing same.

### BACKGROUND OF THE INVENTION

Leaf rust, caused by the fungus Puccinia triticina tritici and stripe (yellow) rust, caused by Puccinia striiformis tritici, are major wheat diseases. Leaf rust and stripe rust cause tremendous yield losses annually. In the last years, stripe rust outbreaks were reported in Australia, China, Pakistan, Central and West Asia, the Middle East (Syria and Turkey), India and U.S.A., indicating virulence changes of the pathogen (Wellings C R et al., 2012. CAB International, pp. 63-83). It was also shown that new stripe rust strains became adapted to higher inoculation temperatures that may account for the hazardous spread of the pathogen (Milus E A et al., 2006. Plant Disease, 90:847-852)

Sharon goatgrass (Aegilops sharonensis Eig) (AES) is a wild diploid (genome S^{sh}S^{sh}; 2n=14) relative of wheat. It is native to the coastal plain of Israel and south Lebanon, growing on stabilized dunes. Work done by Olivera et al. (Olivera P. D. et al. 2007. Plant Disease 91:942-950) on a representative sample of Sharon goatgrass lines collected in Israel and data from the Institute for Cereal Crops Improvement (ICCI, Israel) (Anikster Y. et al. 2005. Plant Disease 89:303-308) revealed that many accessions are highly resistant to inoculation with leaf rust or stripe rust pathogens. A recent evaluation of 1800 newly collected AES accessions at the ICCI confirmed the high frequency of resistance to these diseases in the species. Genetic analysis in a number of these lines (Olivera P. D. et al. 2008. Phytopathology 98:353-358) demonstrated monogenic inheritance of the resistance genes.

Although the Sharon goatgrass Ss^{h} genome is closely related to the B genome of tetraploid and hexaploid wheat, the two genomes cannot be regarded as being homologous. Gene transfer from Sharon goatgrass may therefore be more difficult as compared to transfer from donor species with homologous genomes. Technical problems (e.g. timing of flowering, time of anther dehiscence) and inherent low crossability with wheat result in very low hybrid seed set. Thereafter, pairing and chromosome segment exchange is rare.

Different procedures have been utilized to transfer genes from wild relatives to wheat (e.g. Feldman M. 1983. Acta Biol. Yugoslay. Genet. 15: 145-161; Millet E. 2007. Isr. J. Plant Sci. 55:277-287; Millet E et al., 2007. CAB International pp. 554-563.; Qi L et al., 2007. Chrom. Res. 15:3-19; Kilian B et al., 2011. Aegilops. In Wild Crop Relatives: Genomic and Breeding Resources, Cereals. Edited by C. Kole Springer-Verlag, Berlin

Heidelberg, pp. 1-76), many of which included production of an amphiploid by chromosome duplication of the interspecific hybrid and use of mutants of the Ph genes, which suppresses homoeologous pairing, and particularly the ph1b allele to allow such pairing.

In addition, Sharon goatgrass possesses gametocidal (Gc) genes (Maan S. 1975.

Crop Sci. 15:287-292; Endo T R. 1985. Jpn. J. Genet. 60: 125-135). Only few AES accessions have been used in genetic studies, but all of them showed a gametocidal effect as reflected in the failure to obtain the whole pure series of addition lines of AES. The finding that chromosome 4Ss^{h} was always included in breeding progenies (Zhang H. et al 2001. Theor. Appl. Genet 103:518-525), supports the contention that Gc genes cause preferential transmission of their hosting chromosome. Their presence in a plant is accompanied by chromosome breakage of gametes not carrying the Gc genes, ultimately leading to semi-sterile spikes.

To avoid this gametocidal effect, an “anti-gametocidal” wheat mutant (Gc2^{mut}; Friebe B. et al. 2003. Chromosoma 111:509-517) that confers normal chromosome segregation rather than preferential transmission of the chromosome carrying the gametocidal gene may be used.

Despite its high resistance to different wheat diseases, Sharon goatgrass has hardly been exploited to improve wheat. Marais et al. (Marais G F et al., 2003. S Afr J Plant Soil 20:193-198) have identified potential useful resistance genes in Sharon goatgrass that were introgressed into common wheat chromosomes. In a further work leaf rust and stripe rust resistance genes, designated Lr56/Yr38, were transferred from Sharon goatgrass to chromosome 6A of common wheat (Marais G F et al. 2006. Euphytica 149:373-380). The translocation break occurred in the area of the long arm of wheat chromosome 6A. The Lr56/Yr38 translocation chromosome was found in effect to be most of the Sharon goatgrass chromosome with the terminal segment of its long arm replaced by a corresponding segment of wheat 6AL chromosome. In an attempt to reduce the amount of the transferred chromatin they employed recombination in the absence of the homoeologous pairing suppressor gene, Ph1, and obtained an intercalary sub-telomeric small introgression carrying the Lr56/Yr38 linked genes (Marais G F et al., 2010. Euphytica 171:15-22).

International (PCT) Patent Applications Publication Nos. WO 1995/029238 and WO 1999/045118 disclose genetic sequences which confer or otherwise facilitate disease resistance in plants such as against rust and mildew. The Application provides transgenic plants carrying the subject genetic sequences enabling the generation of disease resistant plants, particularly disease resistant crop varieties.

International (PCT) Patent Applications Publication No. WO 2013/082335 relates to new disease resistant crops and methods of creating new disease resistant crops. Particularly, the Application discloses a wheat genetic line comprising four highly effective disease resistance genes, Lr19, Sr25, Bdv3 and Qfhs.pur-7EL from the wheat-related grasses, Thinopyrum intermedium and Th. Ponticum, all on the long arm of wheat chromosome 7D. The genes are expected to remain in coupling in wheat genetic lines, resulting in wheat genetic lines with reduced susceptibility to yellow dwarf virus, fusarium head blight, stem rust, and leaf rust.

International (PCT) Patent Applications Publication No. WO 2014197505 discloses transgenic wheat 2174 cultivar with increased resistance to diseases caused by foliar pathogens, including leaf rust, stripe rust, stem rust, and powdery mildew, as well as barley yellow dwarf virus and methods for making the transgenic cultivar. The methods involve genetically engineering (transforming) 2174 to overexpress cDNA encoding the resistance gene LR34 in a form that is correctly spliced.

International (PCT) Patent Applications Publication No. WO 2015/036995 to inventors of the present invention discloses introgression of a large segment of chromosome 6S ^{sh} of Aegilops sharonensis Accession TH548, spanning from position 30cM to position 70cM into wheat chromosome 6B, the introgression conferring or enhancing resistance of the wheat plant to leaf rust and/or stripe disease. A paper of inventors of the present invention and co-workers published after the priority date of the present invention describes a shorter segment characterized by SNP markers conferring or enhancing resistance to both leaf rust and strip diseases caused by highly virulent Puccinia races (Khazan Set al.2020. BMC Plant Biology 20(153). doi.org/10.1186/s12870-020-2306-9.

International (PCT) Patent Applications Publication No. WO 2019197408 discloses an isolated nucleic acid encoding a nucleotide-binding and leucine-rich repeat (NLR) polypeptide comprising a zinc-finger BED domain, wherein expression of the NLR polypeptide in a plant confers or enhances resistance of the plant to a fungus, for example wheat yellow (stripe) rust fungus Puccinia striiformis f. sp. tritici.

It is well accepted that using resistant varieties is the most efficient and economical way to control the leaf rust and stripe rust diseases. However, high pace of alterations of the pathogens quickly erodes the primary pool of rust resistance genes. The incidence of severe rust outbreaks has intensified in the past years, primarily due to the appearance of highly virulent races of the pathogen that overcome popular rust resistance sources (Hovmøller M S et al, 2016. Plant Pathol 65:402-411; Liu T et al, 2017. Plant disease 101:1522-1532). This situation is expected to worsen due to further elimination of the remaining sources of rust resistance that have been used in wheat breeding, along with climatic changes that create favorable conditions for disease outbreak (Lyon and Broders, 2017). Hence, new sources of rust resistance are needed to boost the rust resistance gene pool of wheat. In addition, there is a need for resistance-conferring genetic elements that can be introduced into the wheat genome without negatively affecting the wheat growth characteristics and, most importantly, the yield.

Thus, there is a recognized need for and it would be highly advantageous to have genetic elements conferring or enhancing resistance of commercial agricultural wheat cultivars towards rust diseases without negatively affecting the agricultural characteristics of the wheat cultivars.

### SUMMARY OF THE INVENTION

The present invention provides polynucleotide sequences that confers, enhances or otherwise facilitates the resistance of wheat plants and cultivars comprising these sequences in their genome to leaf rust and stripe rust diseases. The present invention further provides wheat plants that are resistant to highly virulent forms of Puccinia fungi inducing leaf rust and stripe rust diseases, including resistant elite wheat cultivars.

The present invention is based in part of the unexpected discovery of a short segment of Aegilops sharonensis chromosome 6S ^{sh} (spanning between about position 34 Mbp to about position 62 Mbp of the 6S^{sh} chromosome), parts of which suffice to enhance resistance to leaf rust and stripe rust diseases. This discovery is of high significance, enabling easier genetic manipulations for transforming the resistance-conferring segment to susceptible plants. The present invention further discloses that inserting the segment or a part thereof to susceptible wheat cultivar, particularly into the wheat chromosome 6B at between about position 33 Mbp to about position 50Mbp results in conferring significant resistance to the wheat to both leaf rust and strip rust diseases without negatively affecting the wheat cultivar phenotype.

According to one aspect, the present invention provides a wheat plant comprising in its genome a heterologous polynucleotide segment conferring or enhancing resistance of the wheat plant to leaf rust and stripe rust diseases, wherein the heterologous polynucleotide segment comprises at least one scaffold having a nucleic acid sequence at least 70% homologous to a nucleic acid sequence set forth in any one of SEQ ID NOs:1-31 or a part thereof. Each possibility represents a separate embodiment of the present invention.

According to certain embodiments, the heterologous polynucleotide segment comprises at least one scaffold having a nucleic acid sequence at least 75%, at least 80%, at least 85%, at least 90% or at least 95% homologous to a nucleic acid sequence set forth in any one of SEQ ID NOs:1-31 or a part thereof. Each possibility represents a separate embodiment of the present invention.

According to certain embodiments, the heterologous polynucleotide segment comprises at least one scaffold having the nucleic acid sequence set forth in any one of SEQ ID NOs:1-31 or part thereof. Each possibility represents a separate embodiment of the present invention.

According to certain exemplary embodiments, the heterologous polynucleotide segment comprises at least one scaffold at least 70% homologous a nucleic acid sequence set forth in any one of SEQ ID NO:1, SEQ ID NO:3, SEQ ID NO:31, SEQ ID NO:23, SEQ ID NO:2 or a part thereof. Each possibility represents a separate embodiment of the present invention.

According to certain embodiments, the heterologous polynucleotide segment comprises at least one scaffold having a nucleic acid sequence at least 70%, at least 75%, at least 80%, at least 85%, at least 90% or at least 95% homologous to a nucleic acid sequence set forth in any one of SEQ ID NO:1, SEQ ID NO:3, SEQ ID NO:31, SEQ ID NO:23, SEQ ID NO:2 or a part thereof. Each possibility represents a separate embodiment of the present invention.

According to certain embodiments, the heterologous polynucleotide segment comprises a scaffold at least 95% homologous to the nucleic acid sequence set forth in SEQ ID NO:1 or a part thereof. According to some embodiments, the heterologous polynucleotide segment comprises a scaffold having the nucleic acid sequence set forth in SEQ ID NO:1.

According to certain embodiments, the heterologous polynucleotide segment comprises a scaffold at least 95% homologous to the nucleic acid sequence set forth in SEQ ID NO:3 or a part thereof. According to some embodiments, the heterologous polynucleotide segment comprises a scaffold having the nucleic acid sequence set forth in SEQ ID NO:3.

According to certain embodiments, the heterologous polynucleotide segment comprises a scaffold at least 95% homologous to the nucleic acid sequence set forth in SEQ ID NO:31 or a part thereof. According to some embodiments, the heterologous polynucleotide segment comprises a scaffold having the nucleic acid sequence set forth in SEQ ID NO:31.

According to certain embodiments the heterologous polynucleotide segment comprises a scaffold at least 95% homologous to the nucleic acid sequence set forth in SEQ ID NO:23 or part thereof. According to some embodiments, the heterologous polynucleotide segment comprises a scaffold having the nucleic acid sequence set forth in SEQ ID NO:23.

According to certain embodiments the heterologous polynucleotide segment comprises a scaffold at least 95% homologous to the nucleic acid sequence set forth in SEQ ID NO:2 or part thereof. According to some embodiments, the heterologous polynucleotide segment comprises a scaffold having the nucleic acid sequence set forth in SEQ ID NO:2.

According to certain embodiments, the length of heterologous polynucleotide segment is in the range of from about 5Kbp to about 28Mbp, from about 20Kbp to about 15Mbp, or from about 50Kbp to about 1Mbp. According to some embodiments, the length of the heterologous polynucleotide segment is from about 5Kbp to about 1Mbp, from about 5Kbp to about 50 Kbp or from about 5Kbp to about 20Kbp. Each possibility represents a separate embodiment of the present invention.

According to certain embodiments, the heterologous polynucleotide segment comprises a fragment of chromosome 6S^{sh} of Aegilops sharonensis Accession TH548, seed of which have been deposited with NCIMB Ltd. as the International Depositary

Authority under Accession No. NCIMB 43567. According to certain embodiments, the fragment comprises a nucleic acid sequence present within Ae. sharonensis chromosome 6S^{sh} from about position 34 Mbp to about position 62 Mbp.

According to certain embodiments, the heterologous polynucleotide segment comprises a nucleic acid marker designated 2-3HS2, wherein the marker is amplified by a pair of primers comprising a forward primer comprising the nucleic acid sequence set forth in SEQ ID NO:32 and a reveres primer comprising the nucleic acid sequence set forth in SEQ ID NO:33.

According to certain exemplary embodiments, the marker comprises a nucleic acid sequence at least 90% homologous to SEQ ID NO:34. According to further certain exemplary embodiments, the marker comprises the nucleic acid sequence set forth in SEQ ID NO:34. According to additional or alternative embodiments, the fragment of the Ae. sharonensis chromosome 6S^{sh} comprises at least one scaffold or a part thereof selected from the group consisting of: scaffold 19799 having the nucleic acid sequence of SEQ ID NO:1 positioned around 58Mbp; scaffold 00757 having the nucleic acid sequence of SEQ ID NO:3 positioned around 57.5Mbp; Scaffold 1934717 having the nucleic acid sequence of SEQ ID NO:31 positioned around 57.7 Mbp; scaffold 1549600 having the nucleic acid sequence of SEQ ID NO:23 positioned around 56Mbp; scaffold 20860 having the nucleic acid sequence of SEQ ID NO:2 positioned around 44Mbp; scaffold 1540406 having the nucleic acid sequence of SEQ ID NO:4 positioned around 32Mbp; scaffold 1900261 having the nucleic acid sequence of SEQ ID NO:5 positioned around 33Mbp; scaffold 1933170 having the nucleic acid sequence of SEQ ID NO:6 positioned around 34Mbp; scaffold 1531163 having the nucleic acid sequence of SEQ ID NO:7 positioned around 35Mbp; scaffold 1927703 having the nucleic acid sequence of SEQ ID NO:8 positioned around 35Mbp; scaffold 1895355 having the nucleic acid sequence of SEQ ID NO:9 positioned around 38Mbp; scaffold 1931208 having the nucleic acid sequence of SEQ ID NO:10 positioned around 38Mbp; scaffold 1935984 having the nucleic acid sequence of SEQ ID NO:11 positioned around 39Mbp; scaffold 1913977 having the nucleic acid sequence of SEQ ID NO:12 positioned around 42Mbp; scaffold 1916860 having the nucleic acid sequence of SEQ ID NO:13 positioned around 43Mbp; scaffold 1891763 having the nucleic acid sequence of SEQ ID NO:14 positioned around 44Mbp; scaffold 1937448 having the nucleic acid sequence of SEQ ID NO:15 positioned around 44Mbp; scaffold 1896571 having the nucleic acid sequence of SEQ ID NO:16 positioned around 46Mbp; scaffold 1528020 having the nucleic acid sequence of SEQ ID NO:17 positioned around 47Mbp; scaffold 1935712 having the nucleic acid sequence of SEQ ID NO:18 positioned around 47Mbp; scaffold 1893110 having the nucleic acid sequence of SEQ ID NO:19 positioned around 48Mbp or around 57Mbp; scaffold 1898148 having the nucleic acid sequence of SEQ ID NO:20 positioned around 51Mbp; scaffold 1934541 having the nucleic acid sequence of SEQ ID NO:21 positioned around 51Mbp; scaffold 1538547 having the nucleic acid sequence of SEQ ID NO:22 positioned around 56Mbp; scaffold 1872406 having the nucleic acid sequence of SEQ ID NO:24 positioned around 56Mbp; scaffold 1889560 having the nucleic acid sequence of SEQ ID NO:25 positioned around 58Mbp; scaffold 75951 having the nucleic acid sequence of SEQ ID NO:26 positioned around 58Mbp; scaffold 1893791 having the nucleic acid sequence of SEQ ID NO:27 positioned around 59Mbp; scaffold 1892710 having the nucleic acid sequence of SEQ ID NO:28 positioned around 61Mbp; scaffold 1926247 having the nucleic acid sequence of SEQ ID NO:29 positioned around 57.5 Mbp; Scaffold 1929618 having the nucleic acid sequence of SEQ ID NO:30 positioned around 57.4 Mbp; and any combination thereof. Each possibility represents a separate embodiment of the present invention.

According to certain exemplary embodiments, the fragment of the Ae. sharonensis chromosome 6S^{sh} comprises at least one of scaffold 19799, scaffold 00757, Scaffold 1934717, scaffold 1549600, scaffold 20860, and fragments thereof. Each possibility represents a separate embodiment of the present invention.

According to certain embodiments, the fragment of the Ae. sharonensis chromosome 6S^{sh} comprises scaffold 20860 located around position 44Mbp of Ae. sharonensis chromosome 6S^{sh}, having the nucleic acid sequence set forth in SEQ ID NO:2 or a part thereof.

According to certain embodiments, the fragment of the Ae. sharonensis chromosome 6S^{sh} comprises scaffold 19799 located around position 58Mbp of Ae. sharonensis chromosome 6S^{sh}, having the nucleic acid sequence set forth in SEQ ID NO:1 or a part thereof.

According to certain embodiments, the fragment of the Ae. sharonensis chromosome 6S′ comprises scaffold 00757 located around position 57.5Mbp of Ae. sharonensis chromosome 6S^{sh}, having the nucleic acid sequence set forth in SEQ ID NO:3 or a part thereof.

According to certain embodiments, the fragment of the Ae. sharonensis chromosome 6S^{sh} comprises scaffold 1934717 located around position 57.7 Mbp of Ae. sharonensis chromosome 6Ss^{h}, having the nucleic acid sequence of SEQ ID NO:31 or a part thereof.

According to certain embodiments, the fragment of the Ae. sharonensis chromosome 6S^{sh} comprises scaffold 1549600 located around position 56Mbp of Ae. sharonensis chromosome 6Ss^{h}, having the nucleic acid sequence of SEQ ID NO:23 or a part thereof.

According to certain embodiments, the heterologous polynucleotide segment is devoid of the nucleic acid sequence set forth in SEQ ID NO:35, SEQ ID NO:36, or a combination thereof.

According to certain embodiments, the heterologous polynucleotide segment is located within chromosome 6B of the wheat plant. According to certain exemplary embodiments, the heterologous polynucleotide segment is located within wheat chromosome 6B at a position between about 33 Mbp to about 50 Mbp.

According to certain embodiments, the heterologous polynucleotide segment is a nucleic acid construct further comprising at least one regulatory element. The nucleic acid construct can be a transformation vector, an expression vector or a combination thereof.

According to certain exemplary embodiments, the wheat plant of the present invention is a cultivar suitable for commercial agricultural growth, but it is not restricted to a specific species, strain or variety. According to certain exemplary embodiments, the wheat cultivar comprising the heterologous polynucleotide segment is of a species selected from the group consisting of Triticum turgidum and Triticum aestivum. According to certain embodiments, the wheat plant is an elite agricultural cultivar.

According to certain embodiments, the wheat plant is homozygous for chromosome 6B comprising the heterologous polynucleotide segment. According to other embodiments, the wheat plant is heterozygous, comprising a native wheat chromosome 6B and chromosome 6B comprising the heterologous polynucleotide segment.

According to certain embodiments, the wheat plant comprising the heterologous polynucleotide segment shows a phenotype of enhanced resistance or tolerance to leaf rust and stripe rust diseases compared to a corresponding plant not comprising within its genome the heterologous polynucleotide segment.

According to some embodiments, the wheat plant comprises the functional homoeologous pairing suppressor gene Ph1. It is to be explicitly understood that the wheat is devoid of the ph1 mutant allele(s).

According to additional embodiments, the wheat plant is devoid of Ae. sharonensis gametocidal Gc2 gene and/or a mutant thereof.

It is to be explicitly understood that the wheat plants and cultivars of the present invention are fertile. Seeds and any other plant part that can be used for propagation, including isolated cells and tissue cultures are also encompassed within the scope of the present invention. It is to be understood that the plant produced from said seeds or other propagating material comprises the heterologous polynucleotide segment that confers or enhances resistance to leaf rust and strip rust diseases as described herein.

According to certain embodiments, leaf rust disease is caused by the fungus Puccinia triticina. According to certain exemplary embodiments, the leaf rust disease is caused by Puccinia triticina tritici. According to certain embodiments, stripe rust disease is caused by the fungus Puccinia striiformis. According to certain exemplary embodiments, the stripe rust disease is caused by Puccinia striiformis tritici.

According to additional aspect, the present invention provides a wheat plant comprising within its genome a heterologous polynucleotide segment comprising at least one scaffold having a nucleic acid sequence at least 70% homologous to a nucleic acid sequence set forth in any one of SEQ ID NOs:1-31 or a part thereof, wherein the polynucleotide confers or enhances resistance of the wheat plant to a disease caused by the fungus Puccinia.

The heterologous polynucleotide segment is as described herein above. According to certain embodiments, the Puccinia is Puccinia triticina and the disease is leaf rust. According to certain embodiments, the Puccinia is Puccinia striiformis and the disease is stripe rust.

According to yet additional aspect the present invention provides a method for producing a wheat plant having enhanced resistance to leaf rust and strip rust diseases, the method comprises introducing into at least one cell of a wheat plant susceptible to the rust diseases a heterologous polynucleotide segment comprising at least one scaffold having a nucleic acid sequence at least 70% homologous to a nucleic acid sequence set forth in any one of SEQ ID NOs:1-31 or a part thereof, thereby producing a wheat plant having enhanced resistance to said rust diseases compared to a corresponding control plant.

According to certain embodiments, the heterologous polynucleotide segment is introduced into chromosome 6B of the at least one cell of the susceptible wheat plant.

According to certain exemplary embodiments, the heterologous polynucleotide segment is introduced into a position within wheat chromosome 6B at between about 33 Mbp to about 50Mbp.

According to certain embodiments, the wheat plant is a wheat cultivar as described hereinabove.

According to certain embodiments, the control plant is a wheat plant or cultivar susceptible to leaf rust and stripe rust disease. According to some embodiments, the control plant is lacking the heterologous polynucleotide segment. According to certain embodiments, the control wheat plant has the same genetic background.

Any method as is known to a person skilled in the art can be used to introduce the heterologous polynucleotide segment of the present invention into a susceptible wheat plant.

According to certain embodiments, the heterologous polynucleotide segment is an isolated polynucleotide or a construct comprising same. According to these embodiments, the heterologous polynucleotide segment is introduced by transforming said isolated polynucleotide or construct comprising same into at least one cell of the susceptible wheat plant. According to certain embodiments, the isolated polynucleotide is introduced by subjecting at least one cell of the susceptible wheat plant to genome editing using artificially engineered nucleases.

According to certain embodiments, the heterologous polynucleotide segment forms part of chromosome 6S^{sh} of Ae. sharonensis. According to certain exemplary embodiments, the Ae. sharonensis is Ae. sharonensis Accession TH548 described hereinabove. According to these embodiments, the heterologous polynucleotide segment is introduced to the susceptible wheat plant without prior isolation by introgression of Ae. sharonensis chromosome 6S^{sh} fragment spanning from about position 34 to about position 64 or a part thereof into the susceptible wheat plant.

According to additional aspect, the present invention provides a method for selecting a wheat plant having an enhanced resistance to leaf rust and stripe rust diseases, comprising the steps of:
a. providing a plurality of plants each comprising at least one cell comprising a heterologous polynucleotide segment conferring or enhancing resistance of the wheat cultivar to leaf rust and stripe rust disease wherein the heterologous polynucleotide segment is at least 70% homologous to a nucleic acid sequence set forth in any one of SEQ ID NOs:1-31 or a part thereof; and
b. selecting plants showing an enhanced resistance to said rust diseases compared to a control wheat plant or to a pre-determined resistance score value;

thereby selecting a plant having enhanced resistance to said rust diseases.

According to certain embodiments, the control plant is a wheat plant susceptible to the rust diseases. According to some embodiments, the susceptible control wheat plant is of the same genetic background.

According to certain embodiments, selecting plants resistant to the rust diseases is performed by inoculating the plants with the respective fungus and selecting phenotypically resistant plants. According to certain exemplary embodiments, the inoculation and selection is performed at the seedling stage of the plants.

The respective fungus is as described hereinabove.

According to additional or alternative embodiments, selecting plants resistant to the rust diseases is performed by detecting the presence of the heterologous polynucleotide segment within the genome of the wheat plant. Any method as is known in the art can be used to detect the heterologous polynucleotide segment. According to certain exemplary embodiments, detection is performed by identifying a sequence of the at least one scaffold located within the heterologous polynucleotide segment described hereinabove.

According to certain embodiments, detection is performed by identifying at least one sequence specific probe that specifically hybridizes under stringent conditions to a nucleic acid sequence at least 70% homologous to any one of SEQ ID NOs:1-31 or a part thereof.

According to certain further exemplary embodiments, detection is performed by identifying the presence of the marker 2-3HS2 comprising a nucleic acid sequence at least 90% homologous to the nucleic acid sequence set forth in SEQ ID NO:34. According to some embodiments, the presence of the marker 2-3HS2 is identified using a pair of primers having the nucleic acid sequence set forth in SEQ ID NO:32 and SEQ ID NO:33.

According to certain embodiments, the plants are further selected to be devoid of the ph1 mutant gene.

According to yet additional aspect, the present invention provides a method for identifying and selecting wheat plants having enhanced resistance or tolerance to leaf rust and stripe diseases, comprising the steps of:
a. providing a plurality of wheat plants;
b. examining a nucleic acid sample obtained from each of the plurality of wheat plants for the presence of a heterologous polynucleotide segment comprising at least one scaffold at least 70% homologous to a nucleic acid sequence set forth in any one of SEQ ID NOs:1-31 or a part thereof; optionally
c. examining a nucleic acid sample obtained from each of the plurality of wheat plants for the presence of a 2-3HS2 marker having a nucleic acid sequence at least 90% homologous to the nucleic acid sequence set forth in SEQ ID NO:34; and
d. selecting wheat plants comprising the heterologous polynucleotide segment and/or the 2-3HS2 marker.

Identifying the heterologous polynucleotide segment and/or 2-3HS2 marker is performed as is known in the Art and as described hereinabove.

According to certain embodiments, the method further comprises inoculating the wheat plants comprising the heterologous polynucleotide segment with the respective fungus and selecting phenotypically resistant plants. According to certain exemplary embodiments, the inoculation and selection is performed at the seedling stage of the plants. The respective fungus is as described hereinabove.

According to yet further aspect, the present invention provides an isolated polynucleotide comprising at least one scaffold having a nucleic acid sequence at least 70% homologous to a nucleic acid sequence set forth in any one of SEQ ID NOs:1-31 or a part thereof, wherein the polynucleotide, when introduced into a wheat plant, confers or enhances resistance of the wheat plant to a leaf rust disease and strip rust disease. Each possibility represents a separate embodiment of the present invention.

According to certain embodiments, the isolated polynucleotide comprises at least one scaffold having a nucleic acid sequence at least 75%, at least 80%, at least 85%, at least 90% or at least 95% homologous to a nucleic acid sequence set forth in any one of SEQ ID NOs:1-31 or a part thereof. Each possibility represents a separate embodiment of the present invention.

According to certain embodiments, the isolated polynucleotide comprises at least one scaffold having the nucleic acid sequence set forth in any one of SEQ ID NOs:1-31 or a part thereof. Each possibility represents a separate embodiment of the present invention.

According to certain exemplary embodiments, the isolated polynucleotide comprises at least one scaffold at least 70% homologous a nucleic acid sequence set forth in any one of SEQ ID NO:1, SEQ ID NO:3, SEQ ID NO:31, SEQ ID NO:23, SEQ ID NO:2 or a part thereof. Each possibility represents a separate embodiment of the present invention.

According to certain embodiments, the isolated polynucleotide comprises at least one scaffold having a nucleic acid sequence at least 70%, at least 75%, at least 80%, at least 85%, at least 90% or at least 95% homologous to a nucleic acid sequence set forth in any one of SEQ ID NO:1, SEQ ID NO:3, SEQ ID NO:31, SEQ ID NO:23, SEQ ID NO:2 or a part thereof. Each possibility represents a separate embodiment of the present invention.

According to certain embodiments, the isolated polynucleotide comprises a scaffold at least 95% homologous to SEQ ID NO:1 or part thereof. According to some embodiments, the isolated polynucleotide comprises a scaffold having the nucleic acid sequence set forth in SEQ ID NO:1.

According to certain embodiments the isolated polynucleotide comprises a scaffold at least 95% homologous to SEQ ID NO:2 or a part thereof. According to some embodiments, the isolated polynucleotide comprises a scaffold having the nucleic acid sequence set forth in SEQ ID NO:2.

According to certain embodiments the isolated polynucleotide comprises a scaffold at least 95% homologous to SEQ ID NO:3 or a part thereof. According to some embodiments, the isolated polynucleotide comprises a scaffold having the nucleic acid sequence set forth in SEQ ID NO:3.

According to certain embodiments, the isolated polynucleotide comprises a scaffold at least 95% homologous to the nucleic acid sequence set forth in SEQ ID NO:31 or a part thereof. According to some embodiments, the isolated polynucleotide comprises a scaffold having the nucleic acid sequence set forth in SEQ ID NO:31.

According to certain embodiments, the isolated polynucleotide comprises a scaffold at least 95% homologous to the nucleic acid sequence set forth in SEQ ID NO:23 or a part thereof. According to some embodiments, the isolated polynucleotide comprises a scaffold having the nucleic acid sequence set forth in SEQ ID NO:23.

According to certain embodiments, the isolated polynucleotide comprises a fragment of chromosome 6S^{sh} of Ae. sharonensis Accession TH548, seed of which have been deposited with NCIMB Ltd. as the International Depositary Authority under Accession No. NCIMB 43567. According to certain embodiments, the isolated polynucleotide comprises a nucleic acid sequence present within Ae. sharonensis chromosome 6S^{sh} from about position of 34 Mbp to about position 62 Mbp.

According to certain embodiments, the isolated polynucleotide comprises a marker designated 2-3HS2, wherein the marker is amplified by a pair of primers comprising a forward primer comprising the nucleic acid sequence set forth in SEQ ID NO:32 and a reveres primer comprising the nucleic acid sequence set forth in SEQ ID NO:33.

According to certain exemplary embodiments, the marker comprises a nucleic acid sequence at least 90% homologous to SEQ ID NO:34. According to further certain exemplary embodiments, the marker comprises the nucleic acid sequence set forth in SEQ ID NO:34.

According to certain embodiments, the isolated polynucleotide is devoid of the nucleic acid sequence set forth in SEQ ID NO:35, SEQ ID NO:36, or a combination thereof.

According to additional aspect, the present invention provides a nucleic acid construct comprising the isolated polynucleotides according to some embodiments of the invention, further comprising at least one regulatory element for directing the expression of the polynucleotide within a plant cell. According to certain embodiment, the regulatory element is selected from the group consisting of a promoter, an enhancer and a translation terminator sequence. The regulatory element, particularly the promoter, can be endogenous or heterologous to the plant comprising the nucleic acid construct. According to certain exemplary embodiments, the plant is a wheat plant.

According to certain exemplary embodiments, leaf rust is caused by the fungus Puccinia triticina. According to certain typical embodiments, the leaf rust disease is caused by Puccinia triticina tritici. According to other embodiments, the stripe rust disease is caused by Puccinia striiformis. According to certain typical embodiments, the stripe rust is caused by Puccinia striiformis tritici.

According to yet further aspect, the present invention provides a pair of primers for identifying resistance or tolerance of a wheat plant to leaf rust and stripe rust diseases, the comprising one primer having the nucleic acid sequence set forth in SEQ ID NO:32 and additional primer having the nucleic acid sequence set forth in SEQ ID NO:33.

According to certain embodiments, the pair of primers amplify leaf rust and stripe rust-resistant marker having a nucleic acid sequence at least 90% homologous to SEQ ID NO:34. According to certain exemplary embodiments, the pair of primers amplify leaf rust and stripe rust-resistant marker having the nucleic acid sequence set forth in SEQ ID NO:34. It is to be understood that any combination of each of the aspects and the embodiments disclosed herein is explicitly encompassed within the disclosure of the present invention.

Other objects, features and advantages of the present invention will become clear from the following description and drawings.

### BRIEF DESCRIPTION OF THE FIGURES

FIG. 1 is a schematic presentation of the procedure for the derivation of secondary and tertiary recombinants. R and r denote for presence or absence of the alien resistance gene(s), respectively. Ph and ph denote for Ph1 and ph1b alleles, respectively. HP -homoeologous pairing. Percentage values are the calculated rate of Galil chromatin.

FIG. 2 shows (—)log P-value of Fisher Exact test conducted on SNPs between Ae. sharonensis and bread wheat in chromosome 6B (based on alignment to Chinese Spring (CS) genome). X-axis represents the position of each SNP (represented by circles) on CS genome. Y-axis is the (—)log P-value of Fisher Exact test. FIG. 2A: Total number of SNPs.

FIG. 2B: Zooming into the area of potential SNPs. SNPs with (—)log p>16 are boxed.

FIG. 3 shows gel electrophoresis representing all of the PCR markers used for assessment of the segment boundaries. Galil is the susceptible elite cultivar (lack of bands represents absence of the Ae. sharonensis segment). Line 42 is one of the primary recombinants that served as a positive control. R-6 is an example to a secondary recombinant with a shortened segment towards the long arm telomere, R-10 is an example to a secondary recombinant that recombined towards the short arm telomere.

FIG. 4 demonstrates the Chromosome 6B constitution according to the analysis with PCR markers 1-9 (Table 3). Wheat lines presented include 12 secondary recombinant lines (designated R-[No.]) , one tertiary recombinant line (P-37), with shorter alien segment than in the primary recombinant line 42, R-1018-8 line that was derived from the cross of R-10 and R-18, and R-1016-10 line that was derived from the cross of R-10 and R-16. Galil is the susceptible elite cultivar without the Ae. sharonensis segment. A segment spanning 0-140 Mb of recombinant chromosome 6B was divided into four regions restricted by markers. Symbols+ and − indicate presence and absence of the markers, respectively; dark gray and light gray colors represent presence or absence of Ae. sharonensis segment, respectively. The boxed (intermediate) region is the alien region present in all of the resistant recombinants. Regions I and II are left extensions (towards the short arm telomere); Regions III and IV are right extensions (towards the long arm telomere).

FIG. 5 shows frequency of occurrence of the PCR markers for assessment of the segment. Frequencies are calculated from 20 candidates that were screened with all of the markers.

FIG. 6 demonstrates the scale of reaction to field inoculation by spraying stripe rust isolate #5006. *VR=very resistant; R=resistant; MS=medium susceptible; S=susceptible; VS=very susceptible.

FIG. 7 shows the structure of a recombinant chromosome 6B.

### DETAILED DESCRIPTION OF THE INVENTION

The present invention discloses novel nucleic acid sequences that confer or enhance resistance of wheat plants to rust diseases, particularly leaf rust disease and strip rust disease, (the latter also known as yellow rust disease), which are caused by fungi of the species Puccinia. The invention further provides wheat plants comprising within the genome heterologous polynucleotide segment comprising the resistance-conferring nucleic acid sequence that show enhanced resistance or tolerance to the fungi, and methods of producing same. The present invention further provides markers for identifying resistance to leaf rust and stripe rust diseases.

### Definitions

The terms “comprise”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a part” with reference to a polynucleotide may include a plurality of polynucleotide parts, including mixtures thereof.

The term “about” as used herein refers to a numeric value±10%.

Whenever a numerical range is indicated herein, it is meant to include any cited numeral (fractional or integral) within the indicated range. The phrases “from” a first indicate number and “to” a second indicate number are meant to include the first and second indicated numbers and all the fractional and integral numerals therebetween.

The term “plant” is used herein in its broadest sense. It also refers to a plurality of plant cells that are largely differentiated into a structure that is present at any stage of a plant's development. Such structures include, but are not limited to, a root, stem, shoot, leaf, flower, petal, fruit, etc. According to certain exemplary embodiments, the term “wheat plant” refers to Triticum turgidum subsp. durum (tetraploid wheat=macaroni wheat) and T. aestivum subsp. aestivum (hexaploid wheat=bread wheat=common wheat) of the tribe Triticeae, family Poaceae (Gramineae).

The term “cultivar” (abbreviation cv.) is used herein to denote a plant having a biological status other than a “wild” status, which “wild” status indicates the original non-cultivated or natural state of a plant or accession. The term “cultivar” (for cultivated plants) includes, but is not limited to, semi-natural, semi-wild, weedy, traditional cultivar, landrace, breeding material, research material, breeder's line, synthetic population, hybrid, founder stock/base population, inbred line (parent of hybrid cultivar), segregating population, mutant/genetic stock, and advanced/improved cultivar. The term as used herein includes registered as well as non-registered lines. Examples of cultivars include such cultivated varieties that belong to the species Triticum turgidum and Triticum aestivum, including, but not limited to, “Chinese Spring” (CS) and “Galil”.

The terms “Aegilops sharonensis” or “Ae. sharonensis” or “AES” are used herein interchangeably and relate to a wild type plant resistant to a disease caused by a fungus of the species Puccinia, particularly by Puccinia triticina and/or Puccinia striiformis. According to certain exemplary embodiments, the term refers to Ae. sharonensis accession TH548 that is resistant to both Puccinia triticina and Puccinia striiformis and is thus resistant to the leaf rust and stripe rust diseases. Seeds of Ae. sharonensis TH548 have been deposited by Ramot at Tel Aviv University Ltd. Israel, a co-manager of the Applicant of the present invention (Technology Innovation Momentum Fund (Israel) Limited Partnership) pursuant to and in satisfaction of the requirement of the Budapest Treaty on the International Recognition of the Deposit of Microorganisms for the Purpose of the Patent Procedure (“The Budapest Treaty”) with the National Collection of Industrial, Food and Marine Bacteria (NCIMB) (NCIMB Ltd, Ferguson Building, Craibstone Estate, Bucksburn, Aberdeen, AB21 9YA Scotland) on Jan. 31, 2020 under Accession No. NCIMB 43567. This accession is also available from the Harold and Adele Lieberman Germplasm Bank at the Institute for Cereal Crops Improvement, Tel Aviv University (http s://www.genesys-pgr.org/wiews/ISR003) under the number AE-548-4.

The terms “resistant” and “resistance” as used herein refer to the ability of a plant variety to restrict the growth and development of a specified pest or pathogen and/or the damage they cause when compared to susceptible plant varieties under similar environmental conditions and pest or pathogen pressure. The terms encompass both partial and full resistance to infection. A rust-resistant plant may either be fully resistant or have low levels of susceptibility to infection by the fungus Puccinia, particularly by Puccinia triticina, and/or Puccinia striiformis, more particularly by Puccinia triticina tritici and/or Puccinia striiformis tritici.

The terms “tolerant” and “tolerance” are used herein to indicate a phenotype of a plant wherein at least some of the disease-symptoms remain absent upon exposure of said plant to an infective dose of a pathogen, particularly fungi, whereby the presence of the pathogen can be established, at least under some culture conditions. Tolerant plants are therefore resistant for symptom expression but symptomless carriers of the pathogen, particularly the fungi.

The terms “susceptible” and “susceptibility” as used herein refer to the inability of a plant to restrict the growth and development of a specified pest or pathogen; a susceptible plant displays the detrimental symptoms linked to the pathogen infection, particularly fungi infection. A rust-susceptible wheat plant may be either non-resistant or have low levels of resistance to these fungi. Stripe rust (also designated yellow rust, caused by the fungus Puccinia striiformis) and leaf rust (caused by Puccinia triticina) are two devastating wheat diseases causing enormous annual yield losses. The fungal pathogens are changing frequently, giving rise to new virulent types and thus overcoming the currently deployed resistance genes. Consequently, the primary wheat gene pool is becoming exhausted and new resistance genes are required. Wild relatives of wheat are yet an untapped resistance gene pool.

Accordingly, “conferred resistance to rust disease(s)” or “enhanced resistance to a rust disease(s)” refer to a phenotype in which a plant, a wheat plant according to the present invention, has greater health, growth, multiplication, fertility, vigor, strength (e.g., stem strength and resistance), yield, or less severe symptoms associated with infection of the pathogenic fungus causing the rust disease compared to a wheat plant that does not have enhanced resistance to the pathogen. Where a plant is tested for resistance, a control plant is used to assess the degree of the plant resistance. According to certain embodiments of the present invention, the control plant is a plant not manipulated to comprise within its genome the resistance-conferring or enhancing polynucleotide segments of the invention. The control plant typically, but not necessarily, has the same genetic background as the examined plant. The enhancement can be manifested as an increase of 0.1%, 0.2%, 0.3%, 0.5%, 0.75%, 1%, 1.5%, 2%, 3%, 4%, 5%, 6%, 7%, 8%, 9%, 10%, 12%, 15%, 17%, 20%, 25%, 30%, 35%, 40%, 50%, 60%, 70%, 80%, 90%, 100%, or more in health, growth, multiplication, fertility, vigor, strength, or yield, as compared to a control plant. The enhancement can be a decrease of 0.1%, 0.2%, 0.3%, 0.5%, 0.75%, 1%, 1.5%, 2%, 3%, 4%, 5%, 6%, 7%, 8%, 9%, 10%, 12%, 15%, 17%, 20%, 25%, 30%, 35%, 40%, 50%, 60%, 70%, 80%, 90%, or 100% in the symptoms associated with the fungi infection as compared to the control plant. According to certain exemplary embodiments, the examined plant and the control plant are grown under the same conditions.

The term “locus” (plural “loci”) as used herein refers to any site that has been defined genetically. The locus can be a single position (nucleotide) or a chromosomal region. A locus may be a gene, a genetic determinant, or part of a gene, or a DNA sequence, and may be occupied by different sequences. A locus may also be defined by an SNP (Single Nucleotide Polymorphism), by several SNPs, or by two flanking SNPs. According to certain embodiments, locus is defined herein as the position that a given gene or genetic determinant occupies on a chromosome of a given species.

As used herein the term “polynucleotide” refers to a single or double stranded nucleic acid sequence which is isolated and provided in the form of an RNA sequence, a complementary polynucleotide sequence (cDNA), a DNA sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

The term “isolated” refers to at least partially separated from the natural environment e.g., from a plant cell.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

The term “heterologous” with reference to a polynucleotide as is used herein refers to a sequence that is not naturally found in the plant, specifically the wheat plant, and has been artificially introduced into the plant.

The term “heterozygous” as is used herein means a genetic condition existing when different alleles (forms of a given gene, genetic determinant or sequences) reside at corresponding loci on homologous chromosomes.

The term “homozygous” as is used herein, means a genetic condition existing when identical alleles (forms of a given gene, genetic determinant or sequences) reside at corresponding loci on homologous chromosomes.

The terms “introgression” “introgressed” and “introgressing” refer to the translocation of a desired allele(s) (forms of a given gene, genetic determinant or sequences) from a genetic background of one species, variety or cultivar into the genome of another species, variety or cultivar. In one method, the desired allele(s) can be introgressed through a sexual cross between two parents, wherein one of the parents has the desired allele in its genome. The desired allele can include desired gene or genes, a marker locus, a QTL or the like.

The terms “genetic engineering”, “transformation” and “genetic modification” are all used herein for the transfer of isolated and cloned genes, genetic determinants or polynucleotides into the DNA, usually the chromosomal DNA or genome, of another plant, or to the modification of a gene within the plant genome.

As used herein, the term “plant part” typically refers to a part of the wheat plant, including single cells and cell tissues such as plant cells that are intact in plants, cell clumps and tissue cultures from which wheat plants can be regenerated. Examples of plant parts include, but are not limited to, single cells and tissues from pollen, ovules, leaves, embryos, roots, root tips, anthers, flowers, fruits, stems shoots, and seeds; as well as pollen, ovules, leaves, embryos, roots, root tips, anthers, flowers, fruits, stems, shoots, scions, rootstocks, seeds, protoplasts, calli, and the like.

As used herein, the term “population” refers to a genetically heterogeneous collection of plants sharing a common genetic derivation.

The term “linkage group” as used herein refers to all of the genes or genetic traits that are located on the same chromosome. Within the linkage group, those loci that are close enough together will exhibit linkage in genetic crosses. Since the probability of crossover increases with the physical distance between genes on a chromosome, genes whose locations are far remoted from each other within a linkage group may not exhibit any detectable linkage in direct genetic tests.

The term “marker” as used herein refers to a nucleic acid sequence the presence of which is indicative for a trait, particularly resistance to at least one of strip rust and leaf rust disease.

As used herein, the term “contig” refers to a set of overlapping DNA segments that together represent a consensus region of DNA.

As used herein, the term “scaffold” refers to the order and orientation of adjacent contigs connected together, which can be generally positioned on a target draft genome or a chromosome.

The present invention discloses hitherto unidentified sequences which, when present in the genome of a wheat plant confer or enhance the tolerance and/or resistance of the wheat plant to infection by Puccinia fungi, particularly Puccinia triticina and Puccinia striiformis causing leaf rust disease and stripe rust disease, respectively.

According to certain aspects, the present invention provides isolated polynucleotide comprising a nucleic acid sequence capable of conferring or enhancing resistance to a plant, particularly wheat plant, towards a disease cause by at least one fungus of the species Puccinia, particularly towards leaf rust disease and stripe rust disease.

According to certain embodiments, the isolated polynucleotide comprises at least one scaffold having a nucleic acid sequence at least 70% homologous to a nucleic acid sequence set forth in any one of SEQ ID NOs:1-31 or a part thereof. Each possibility represents a separate embodiment of the present invention.

The at least one scaffold or a combination of scaffold may include a single resistance/tolerance-conferring gene or a plurality of resistance/tolerance-conferring genes, typically two or three genes. According to some embodiments, the at least one scaffold comprises a single resistance/tolerance-conferring gene. According to some embodiments, the at least one scaffold comprises two resistance/tolerance-conferring genes. According to some embodiments, the at least one scaffold comprises three resistance/tolerance-conferring genes.

According to certain embodiments, the isolated polynucleotide comprises at least one scaffold having a nucleic acid sequence at about least 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or more homologous to, or identical to a polynucleotide having a nucleic acid sequence selected from the group consisting of SEQ ID NOs:1-31 and a part or parts thereof. Each possibility represents a separate embodiment of the present invention.

According to certain exemplary embodiments, the isolated polynucleotide comprises a scaffold having a nucleic acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or more homologous to, or identical to the nucleic acid sequence set forth in SEQ ID NO:1.

According to certain additional or alternative exemplary embodiments, the isolated polynucleotide comprises a scaffold having a nucleic acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or more homologous to, or identical to the nucleic acid sequence set forth in SEQ ID NO:2.

According to certain further additional or alternative exemplary embodiments, the isolated polynucleotide comprises a scaffold having a nucleic acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or more homologous to, or identical to the nucleic acid sequence set forth in SEQ ID NO:3.

According to certain further additional or alternative exemplary embodiments, the isolated polynucleotide comprises a scaffold having a nucleic acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or more homologous to, or identical to the nucleic acid sequence set forth in SEQ ID NO:23.

According to certain further additional or alternative exemplary embodiments, the isolated polynucleotide comprises a scaffold having a nucleic acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or more homologous to, or identical to the nucleic acid sequence set forth in SEQ ID NO:31.

As used herein, the terms “homology” or “homologous” when used in relation to nucleic acid sequences refers to a degree of similarity or identity between at least two nucleotide sequences. There may be partial homology or complete homology (i.e., identity). “Sequence identity” refers to a measure of relatedness between two or more nucleotide sequences, expressed as a percentage with reference to the total comparison length. The identity calculation takes into account those nucleotide residues that are identical and in the same relative positions in their respective sequences. A gap, i.e. a position in an alignment where a residue is present in one sequence but not in the other is regards as a position with non-identical residues. Identity (e.g., percent homology) can be determined using any homology comparison software, including for example, the BlastN software of the National Center of Biotechnology Information (NCBI) such as by using default parameters. A widely used and accepted computer program for performing sequence alignments is CLUSTALW v1.6 (Thompson, et al. Nucl. Acids Res., 22: 4673-4680, 1994).

According to some embodiments of the invention, the homology or identity is a global homology or identity, i.e., over the entire nucleic acid sequences of the invention and not over portions thereof.

According to some embodiments of the invention, the homology or identity is a partial homology or identity, i.e., over fragment or fragments of the nucleic acid sequences of the invention and not over portions thereof.

According to certain exemplary embodiments, the isolated polynucleotide comprises a nucleic acid sequence at least 80% homologous to at least one of the fragments of any one of SEQ ID NOs:1-31 described in Table 1.

According to some embodiments, the isolated polynucleotide comprises a nucleic acid sequence at least at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or more homologous to, or identical to at least one of the fragments of any one of SEQ ID NOs:1-31 described in Table 1.

TABLE 1
Scaffold fragments

| SEQ | | | |
|---|---|---|---|
| ID | No. of | | Scaffold fragments |
| NO. | Nucleotides | Scaffold | (nucleobase position from-to) |
| 1 | 28828 | 19799 | 291-330; 478-857; 959-1448; 1628-1732; |
| | | | 1841-6253; 6465-13113; 13149-14038; 14083-14950; |
| | | | 15038-15573; 15634-16457; 166552-16597; |
| | | | 16643-16659; 16835-17147; 17180-19854; |
| | | | 19945-20514; 20567-22596; 22633-27260; |
| 2 | 19882 | 20860 | 110-4174; 4896-7136; 7360-10600; |
| | | | 10643-10860; 10906-11092; 11137-14247; |
| | | | 14312-14653; 14772-15293; 15333-15543; |
| | | | 15579-16082; 16168-16851- |
| 3 | 26399 | 00757 | 1079-1094; 6459-7118; 7191-7931; 7954-12776; |
| | | | 12811-13080; 13183-13240; 13345-16855; |
| | | | 16914-19968; 20445-20575; 20758-22818; |
| | | | 23733-23919; |
| 4 | 64196 | 1540406 | 1; 738-826; 1239-1248; 2320-2399; 2436-2823; |
| | | | 2948-3405; 3491-4095; 4157-4174; 4239-7163; |
| | | | 7393-8012; 8563-9342; 9526-9531; 10062-10109; |
| | | | 10239-12334; 12507-12649; 12787-13385; |
| | | | 13498-14330; 14457-16953; 16994-18526; |
| | | | 18845-19628; 19692-20672; 20714-21774; |
| | | | 21818-23800; 23834-26244; 26291-26591; |
| | | | 26837-29509; 29542-29695; 29843-30051; |
| | | | 30214-30429; 32352-34537; 34557-35059; |
| | | | 35345-35698; 35878-36552; 36572-37427; |
| | | | 37584-37776; 37820-39948; 40034-43399; |
| | | | 43677-43877; 43929-44013; 44472-44751; |
| | | | 44782-44802; 46075-48084; 48124-48289; |
| | | | 48343-50459; 50486-52590; 54252-54350; |
| | | | 54447-54912; 55423-55925; 55992-56176; |
| | | | 56226-58359; 58559-58746; 58849-59139; |
| | | | 59184-59215; 59237-61628; 61731-62781; |
| | | | 62894-63768; 63886-63887; |
| 5 | 24799 | 1900261 | 1-2999; 5310-5678; 5892-5955; 6310-6330; |
| | | | 6758-7383; 7454-8049; 8126-9890; |
| | | | 10508-10768; 10931-11781; 11930-14726; |
| | | | 14787-14804; 15282-16578; 16610-17062; |
| | | | 17273-17354; 17546-21962; 22065-23900; |
| | | | 23941-24196; 24237-24799; |
| 6 | 29906 | 1933170 | 1-29906; |
| 7 | 16584 | 1531163 | 1-16584 |
| 8 | 61472 | 1927703 | 1-61472; |
| 9 | 16716 | 1895355 | 1-16716; |
| 10 | 45874 | 1931208 | 1-45874; |
| 11 | 100249 | 1935984 | 1-5; 5497-5501; 5522-5531; 6073-6713; 6927-7756; |
| | | | 9915-9930; 28825-29705; 29894-29900; |
| | | | 30929-31038; 31444-31823; 31878-33241; |
| | | | 33395-33553; 33585-33652; 33749-34056; |
| | | | 35840-35933; 36121-36363; 36890-37063; |
| | | | 42170-42188; 52442-53045; 56194-56201; |
| | | | 56343; 56706-59540; 60043-60051; |
| | | | 62750-62788; 63283-63291; 64905-67403; |
| | | | 67496-67561; 68325-68335; 69420-69794; |
| | | | 76649-76653; 79298-79382; 79461-79462; |
| | | | 82841-83677; 83797-83922; 84479-84639; |
| | | | 84764-85724; 85981-87197; 87232-87350; |
| | | | 88138-88314; 88788-88857; 89057-89507; |
| | | | 89549-90805; 90839-90897; 91155-91734; |
| | | | 92685-92859; 92988-93109; 96532-96533; |
| | | | 96584-96606; 98934-98942; |
| 12 | 35574 | 1913977 | 2165-2182; 2526-2904; 3074-3300; 3838-4205; |
| | | | 4269-7648; 7706-17555; 17677-17872; |
| | | | 17922-17932; 17987-18238; 18293-21369; |
| | | | 21550-21585; 21795-22226; 22277-22366; |
| | | | 22533-22705; 23199-23283; 23571-23920; |
| | | | 24003-27603; 27714-29575; 29677-31996; |
| | | | 32056-32117; 32150-32556; 32608-33024; |
| 13 | 13004 | 1916860 | 1-13004; |
| 14 | 32429 | 1891763 | 1-7; 325-2902; 2969-3595; 3695-5693; |
| | | | 5828-6622; 6652-6729; 6766-6992; 7049-7054; |
| | | | 7112-7816; 7853-7934; 7996-8297; 8334-13023; |
| | | | 13065-19478; 19511-20189; 20285-20694; |
| | | | 20797-21785; 21882-22196; 22299-22912; |
| | | | 22941-26167; 26219-26866; 26923-27449; |
| | | | 27572-27633; 27653-27795; 27987-28094; |
| | | | 28155-29348; 29433-30333; 30451-30522; |
| | | | 31135-31408; |
| 15 | 32664 | 1937448 | 1-32664; |
| 16 | 26451 | 1896571 | 1-26451; |
| 17 | 22991 | 1528020 | 148-1078; 1340-3164; 3837-5408; 5940-7548; |
| | | | 8003-8244; 8404-11132; 11235-11372; |
| | | | 12999-13467; 14827-14976; 15111-17245; |
| | | | 17352-20327; 22983-22991; |
| 18 | 23698 | 1935712 | 1-23698; |
| 19 | 27610 | 1893110 | 1-27610; |
| 20 | 53213 | 1898148 | 756-1849; 1975-2104; 2165-3864; 3909-4581; |
| | | | 4674-6767; 6797-6801; 7063-7254; 7281-7413; |
| | | | 7583-8860; 8900-9127; 9896-9919; 9981-9984; |
| | | | 10126-12906; 12955-13271; 13302-14855; |
| | | | 15680-15951; 22309-22597; 22820-23781; |
| | | | 24149-24537; 24635-24644; 25729-25860; |
| | | | 26057-27755; 27819-27940; 28006-29772; |
| | | | 30040-30066; 30096-30183; 30586-30760; |
| | | | 30837-33484; 33514-35133; 35173-42000; |
| | | | 42140-44771; 44810-45946; 45967-46403; |
| | | | 47444-47481; 47830-49800; 49849-50631; |
| | | | 50719-51126; 51215-53213; |
| 21 | 75783 | 1934541 | 1-1865; 1920-3105; 3630-3640; 4645-4688; |
| | | | 4898-5878; 6083-8787; 8831-10936; |
| | | | 11060-12461; 12510-12676; 12784-14882; |
| | | | 14956-15183; 17408; 23635-23733; 28519-28528; |
| | | | 29522-29608; 30124-30165; 34475-34524; |
| | | | 34587-34732; 37615-38233; 38797-39011; |
| | | | 41507-41520; 59519-59541; 59599-59755; |
| | | | 59881-62064; 66993-70274; 70398-71016; |
| | | | 71050-73615; |
| 22 | 15026 | 1538547 | 1-3102; 3153-8086; 8216-11267; 11387-11660; |
| | | | 12105-12415; 12604-14106; 14912-15026; |
| 23 | 4667 | 1549600 | 1-4667; |
| 24 | 28545 | 1872406 | 1-28545; |
| 25 | 34868 | 1889560 | 1-34868; |
| 26 | 17615 | 75951 | 341-478; 558-688; 1173-2112; 2176-4336; |
| | | | 4430-4743; 4767-6347; 6470-11080; 11123-11917; |
| | | | 11928-11991; 12171-12178; 12202-13713; |
| | | | 13813-14247; 14275-16857; 16934-17467; |
| | | | 17498-17615; |
| 27 | 29225 | 1893791 | 1-29225; |
| 28 | 15139 | 1892710 | 1-15139; |
| 29 | 22585 | 1926247 | 6069-8437; 8478-10521; 10599-11268; |
| | | | 11376-11542; 11738-11741; 11819-12311; |
| | | | 12331-15430; 15468-19621; 19679-19882; |
| | | | 20090-20447; 22274-22274; 22440-22585; |
| 30 | 20160 | 1929618 | 1; 547-617; 1260-2050; 2381-4139; 4250-5604; |
| | | | 5723-6202; 6278-6338; 6374-8991; 9101-9404; |
| | | | 9628-11264; 11300-17896; |
| 31 | 65001 | 1934717 | 3633-3650; 3837-4674; 7565-7586; 15846-15867; |
| | | | 18640-18641; 20604-20787; 27833-27848; |
| | | | 27887-29053; 30804-30908; 31365-32054; |
| | | | 32103-32855; 32902-33416; 33465-33475; |
| | | | 33499-33767; 33987-34455; 34498-35683; |
| | | | 35717-37244; 37382-37862; 37930-38200; |
| | | | 38345-40086; 40123-40773; 40943-40945; |
| | | | 41106-46396; 46433-46586; 46613-53816; |
| | | | 53888-54137; 54389-56432; 56905-57322; |
| | | | 57354-57889; 57921-57936; 57994-58975; |
| | | | 59142-61925; 61991-63602; 63625-65001; |

According to certain embodiments, the length of isolated polynucleotide is in the range of from about 5Kbp to about 28Mbp, from about 5Kbp to about 15Mbp, from about 5Kbp to about 13.6Mbp, from about 5Kbp to about 1Mbp, from about 20Kbp to about 28Mbp, from about 20Kbp to about 15Mbp, from about 20Kbp to about 13.6Mbp, from about 20Kbp to about 1Mbp, from about 50Kbp to about 28Mbp, from about 50Kbp to about 15Mbp, from about 50Kbp to about 13.6Mbp, from about 50Kbp to about 1Mbp. According to some embodiments, the length of the isolated polynucleotide is from about 5Kbp to about 1Mbp, from about 5Kbp to about 50 Kbp or from about 5Kbp to about 20Kbp. Each possibility represents a separate embodiment of the present invention.

According to certain embodiments, the isolated polynucleotide comprises a nucleic acid sequence of a fragment of chromosome 6S^{sh} of Aegilops sharonensis Accession TH548 described hereinabove. According to certain embodiments, the isolated polynucleotide comprises a nucleic acid sequence present within Ae. sharonensis chromosome 6S^{sh} from position 34 Mbp to position 62 Mbp. It is to be explicitly understood that the isolated polynucleotide of the present invention may include the entire fragment of chromosome Ae. sharonensis 6S^{sh} spanning from position 34 Mbp to position 62 Mbp or fragments thereof.

According to certain exemplary embodiments, the isolated polynucleotide comprises a marker designated 2-3HS2, wherein the marker is amplified by a pair of primers comprising a forward primer comprising the nucleic acid sequence set forth in SEQ ID NO:32 and a reveres primer comprising the nucleic acid sequence set forth in SEQ ID NO:33.

According to certain exemplary embodiments, the marker comprises a nucleic acid sequence at least 90% homologous to SEQ ID NO:34. According to further certain exemplary embodiments, the marker comprises a nucleic acid sequence at least about 91%, at least about 92%, at least about 93%, at least about 94%, at least about 95%, at least about 96%, at least about 97%, at least about 98%, at least about 99% or more homologous to, or identical to the nucleic acid sequence set forth in SEQ ID NO:34.

According to additional aspect, the present invention provides a nucleic acid construct comprising the isolated polynucleotide of the invention, further comprising at least one regulatory element for directing transcription of the nucleic acid sequence in the host plant cell.

According to certain embodiments, the regulatory element is selected from the group consisting of an enhancer, a promoter, a translation termination sequence and the like. According to some embodiments of the invention, the regulatory sequence is operably linked to the isolated polynucleotide.

A nucleic acid sequence (particularly a coding nucleic acid sequence) is “operably linked” to a regulatory sequence (e.g., promoter) if the regulatory sequence is capable of exerting a regulatory effect on the coding sequence linked thereto.

According to certain embodiments, the nucleic acid construct is an expression vector comprising a promoter operably linked to the polynucleotide of the invention.

As used herein, the term “promoter” refers to a region of DNA placed upstream of the transcriptional initiation site of a gene to which RNA polymerase binds to initiate transcription of RNA. The promoter controls where (e.g., which portion of a plant) and/or when (e.g., at which stage or condition in the lifetime of an organism or a cell thereof) the gene is expressed.

According to some embodiments of the invention, the promoter is heterologous to the isolated polynucleotide and/or to the host cell.

As used herein the phrase “heterologous promoter” refers to a promoter from a different species or from the same species but from a different gene locus as of the isolated polynucleotide sequence.

Any suitable promoter sequence can be used by the nucleic acid construct of the present invention. Preferably the promoter is selected from the group consisting of a constitutive promoter, a tissue-specific, or biotic-stress specific promoter, particularly promoters inducible by fungi infection. According to some embodiments of the invention, the promoter is a plant promoter suitable for expression of the isolated polynucleotide of the invention in a wheat plant cell.

Suitable promoters for use in wheat include, but are not limited to the promoter of wheat Lr67 gene (Moore, J., et al. 2015. Nat Genet 47, 1494-1498. Doi:10.1038/ng.3439; the promoter of wheat Lr21 gene (Huang 1 et al., 2003. Genetics 164(2):655-664); the promoter of wild wheat Yr36 gene (Fu D. et al., 2009. Science 323(5919):1357-1360. Doi:10.1126/science.1166289) and the constitutive cauliflower mosaic virus 35S promoter (Odell J T et al., Nature 313:810-812, 1985).

The nucleic acid construct of the present invention can further comprise at least one marker (reporter) gene, operably linked to a regulatory element (such as a promoter) that allows transformed cells containing the marker to be either recovered by negative selection (by inhibiting the growth of cells that do not contain the selectable marker gene), or by positive selection (by screening for the product encoded by the markers gene). Many commonly used selectable marker genes for plant transformation are known in the art, and include, for example, genes that code for enzymes that metabolically detoxify a selective chemical agent which may be an antibiotic or an herbicide, or genes that encode an altered target which is insensitive to the inhibitor. Several positive selection methods are known in the art, such as mannose selection. Alternatively, marker-less transformation can be used to obtain plants without mentioned marker genes, the techniques for which are known in the art.

The construct according to the present invention being a transformation vector, an expression vector or a combination thereof can be, for example, plasmid, a bacmid, a phagemid, a cosmid, a phage, a virus or an artificial chromosome.

The polynucleotides of the invention and construct comprising same can be chemically synthesized by any method as is known in the Art.

The nucleic acid construct comprising the polynucleotide conferring or enhancing resistance to rust diseases as disclosed herein may be used for the production of a wheat plant having enhanced resistance or tolerance to leaf rust disease and stripe rust disease.

According to certain embodiments, the resistance-conferring polynucleotide or the construct comprising same is introduced into a susceptible wheat plant, typically to a wheat cultivar used in agriculture.

The resistance conferring nucleic acid sequence may be introduced to a recipient wheat plant by any method as is known to a person skilled in the art. According to certain embodiments, the isolated polynucleotide or the construct comprising same according to the teachings of the invention can be introduced by transformation. Transformation is optionally followed by selection of offspring plants comprising the resistance-conferring sequence and exhibiting resistance to the fungal diseases stripe rust and leaf rust.

According to certain embodiments, the present invention provides a method for producing a wheat plant having enhanced resistance to leaf rust and strip rust diseases, the method comprises introducing into at least one cell of a wheat plant susceptible to the rust diseases a heterologous polynucleotide segment comprising at least one scaffold having a nucleic acid sequence at least 70% homologous to a nucleic acid sequence set forth in any one of SEQ ID NOs:1-31 or a part thereof, thereby producing a wheat plant having enhanced resistance to said rust diseases compared to a corresponding control plant.

The heterologous polynucleotide segment or a construct comprising same is as described hereinabove.

Methods for transforming a plant cell with nucleic acids sequences according to the present invention are known in the art. As used herein the term “transformation” or “transforming” describes a process by which a foreign nucleic acid sequence, such as a vector, enters and changes a recipient cell into a transformed, genetically modified or transgenic cell. Transformation may be stable, wherein the nucleic acid sequence is integrated into the plant genome and as such represents a stable and inherited trait, or transient, wherein the nucleic acid sequence is expressed by the cell transformed but is not integrated into the genome, and as such represents a transient trait. According to typical embodiments the nucleic acid sequences of the present invention are stably transformed into a plant cell.

There are various methods of introducing foreign nucleic acid sequences into both monocotyledonous and dicotyledonous plants (for example, Potrykus I. 1991. Annu Rev Plant Physiol Plant Mol Biol 42:205-225; Shimamoto K. et al., 1989. Nature 338:274-276).

The principal methods of the stable integration of exogenous DNA into plant genomic DNA includes two main approaches:

Agrobacterium-mediated gene transfer: The Agrobacterium-mediated system includes the use of plasmid vectors that contain defined DNA segments which integrate into the plant genomic DNA. Methods of inoculation of the plant tissue vary depending upon the plant species and the Agrobacterium delivery system. A widely used approach is the leaf-disc procedure, which can be performed with any tissue explant that provides a good source for initiation of whole-plant differentiation (Horsch et al., 1988. Plant Molecular Biology Manual A5, 1-9, Kluwer Academic Publishers, Dordrecht). A supplementary approach employs the Agrobacterium delivery system in combination with vacuum infiltration. Agrobacterium mediated transformation protocols for wheat are known to a person skilled in the art. High efficiency wheat transformation mediated by Agrobacterium tumefaciens is described by Ishida et al. (Ishida Y., et al. In: Ogihara Y., Takumi S., Handa H. (eds) Advances in Wheat Genetics: From Genome to Field.

Springer, Tokyo. DOI 10.1007/978-4-431-55675-6 18).Direct nucleic acid transfer: There are various methods of direct nucleic acid transfer into plant cells. In electroporation, protoplasts are briefly exposed to a strong electric field, opening up mini-pores to allow DNA to enter. In microinjection, the nucleic acid is mechanically injected directly into the cells using micropipettes. In microparticle bombardment, the nucleic acid is adsorbed on microprojectiles such as magnesium sulfate crystals or tungsten particles, and the microprojectiles are physically accelerated into cells or plant tissues. Another method for introducing nucleic acids to plants is via the sonication of target cells. Alternatively, liposome or spheroplast fusion has been used to introduce expression vectors into plants.

Following transformation of wheat target tissues, expression of the above described selectable marker genes allows for preferential selection of transformed cells, tissues and/or plants, using regeneration and selection methods now well known in the art.

According to other embodiments, the resistance conferring polynucleotides of the present invention can be introduced into the genome of a susceptible wheat cultivar using the techniques of genome editing. These techniques are particularly useful for introducing the resistance-conferring polynucleotide into a pre-determined location within chromosome 6B of the susceptible wheat.

Genome editing is a reverse genetics method which uses artificially engineered nucleases to cut and create specific double-stranded breaks at a desired location(s) in the genome, which are then repaired by cellular endogenous processes such as, homology directed repair (HDR) and non-homologous end-joining (NHEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

As described hereinabove, the nucleic acids sequences conferring or enhancing resistance to the rust diseases according to the teachings of the present invention have been discovered by the inventors of the present invention on a fragment of chromosome 6S^{sh} of Aegilops sharonensis Accession TH548 spanning from position 34 Mbp to position 62 Mbp.

Thus, according to alternative embodiments, the resistance-conferring polynucleotide may be translocated without prior isolation from the resistant Ae.

sharonensis plant, by introgression of the Ae. sharonensis chromosome fragment into a wheat plant, preferably into wheat cultivar used for agricultural commercial growth.

The Ae. sharonensis chromosome fragment is as described hereinabove.

Plants resistant to leaf rust and strip rust disease can be selected by examining the presence of nucleic acid sequences of the at least one scaffold and/or markers as described herein above.

According to certain aspects, the present invention provides a wheat plant comprising in its genome a heterologous polynucleotide segment conferring or enhancing resistance of the wheat plant to leaf rust and stripe rust diseases, wherein the heterologous polynucleotide segment comprises at least one scaffold having a nucleic acid sequence at least 70% homologous to a nucleic acid sequence set forth in any one of SEQ ID NOs:1-31 or a part thereof. Each possibility represents a separate embodiment of the present invention.

The heterologous polynucleotide segment and its origin is as described hereinabove.

According to yet additional aspect, the present invention provides a method for identifying and selecting wheat plants having enhanced resistance to leaf rust and stripe resistance, comprising the steps of:
a. providing a plurality of wheat plants;
b. examining a nucleic acid sample obtained from each of the plurality of wheat plant for the presence of a heterologous polynucleotide segment comprising at least one scaffold at least 70% homologous to a nucleic acid sequence set forth in any one of SEQ ID NOs:1-31or a part thereof; optionally
c. examining a nucleic acid sample obtained from each of the plurality of wheat plant for the presence of a marker having the nucleic acid sequence set forth in SEQ ID NO:34; and

selecting wheat plants comprising the heterologous polynucleotide segment.

This method can be defined as “a marker assisted selection” as the selection of the desired resistant phenotype is performed using nucleic acid markers specific for the resistance-conferring nucleic acid sequence. Such marker assisted selection is of particular advantage, enabling selecting resistant platelets at commercial breeding.

According to certain exemplary embodiments, the step of examining a nucleic acid sample obtained from each of the plurality of wheat plants for the presence of the resistance-conferring or enhancing polynucleotide comprise the use of a set of bi-directional primers. Bi-directional means that the orientation of the primers is such that one functions as the forward and one as the reverse primer in an amplification reaction of nucleic acid. The bi-directional primers are typically used in an amplification reaction on genomic DNA that amplifies a unique nucleic acid sequence of the resistance-conferring or enhancing polynucleotide or a marker thereof but that does not amplify wild type sequences. According to certain embodiments, the pair of primers is designed to amplify the marker 2-3HS2 comprising the nucleic acid sequence set forth in SEQ ID NO:34. According to certain exemplary embodiments, the marker 2-3HS2 is amplified by a pair of primer comprising the nucleic acid sequence set forth in SEQ ID NO:32 and SEQ ID NO:33.

Additionally, or alternatively, the markers are sequence specific probes that specifically hybridize under stringent conditions to a nucleic acid sequence at least 70% homologous to any one of SEQ ID NOs:1-31 or a part thereof, but not to a nucleic acids isolated from wheat plant susceptible to leaf rust and/or stripe rust disease, and that can be detected thereafter by various methods as are well known to a person skilled in the art.

Nevertheless, it is to be explicitly understood that the method aspects of the invention are not limited to the use of the markers identified herein, and that methods of the present invention may also make use of markers not explicitly disclosed herein or even yet to be identified, as identifying and using such markers is well within the skills of a person with knowledge in the Art.

In an additional or alternative method, each of the plurality of the wheat plant is phenotypically examined for tolerance or resistance to infection by Puccinia fungi causing leaf rust or strip rust disease as exemplifies hereinbelow.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### Examples

Materials and Methods

Plant Material

The rust resistant primary recombinants were produced using phib induction of homoeologous pairing between wheat cv. Chinese Spring and Aegilops sharonensis (Sharon goatgrass) chromosomes followed by backcrossing to the recurrent wheat parent cv. Galil (Millet et al. 2014. Genome 57: 309-316. dx.doi.org/10.1139/gen-2014-0004). Ae. sharonensis accession TH548, seed of which have been deposited with NCIMB Ltd. as the International Depositary Authority under Accession No. NCIMB 43567 was used. The recombinant source lines used in the present invention and their 6B chromosome constitution are presented in Table 2.

The homoeologous pairing ph1b mutant (HP) in the genetic background of cv. Chinese Spring (CS) was originally obtained from the late E.R. Sears.

Wheat cv. Galil is an elite Israeli spring wheat cultivar bred by Hazera Seed of Growth (Israel). This cultivar possesses the leaf rust resistance Lr26 and stripe rust resistance Yr19 genes, but is susceptible to the leaf and the stripe rust isolates that were used in this research.

TABLE 2
Source lines for the production of secondary recombinant plants

| Primary recombinant lines | | Selected BC₁ | | Number of |
|---|---|---|---|---|
| | Introgression size | (to the HP mutant) | | Fl seed |
| Line | on chromosome | Inoculating | Infection | (BC₁ crossed |
| designation | 6B* (cM) | pathogen | Type (IT)** | with Galil)*** |
| RY-32-3-3 | 30 (7)-107 (120) | Leaf rust | 1 | 44 |
| RL-17-6-10 | 33 (7)-107 (120) | Leaf rust | 1 | 380 |
| RY-41-1-14 | 30 (7)-107 (120) | Leaf rust | 1 | 136 |
| RY-32-3-14 | 33 (13)-87 | Leaf rust | 3-5 | 33 |
| RY-14-1-7 | 38-87 | Leaf rust | 1 | 37 |
| RY-32-3-3 | 30 (7)-107 (120) | Stripe rust | 1 | 116 |
| RL-17-6-10 | 33 (7)-107 (120) | Stripe rust | 1 | 234 |
| RY-32-3-14 | 33 (13)-87 | Stripe rust | 1 | 362 |
| RY-14-1-7 | 38-87 | Stripe rust | 1 | 58 |

*Numbers are based on presence of Ae. sharonensis DArT markers or absence of wheat markers. Numbers in brackets are based on absence of Galil markers in the recombinant line and in CS (Millet et al. 2014, ibid). RY and RL denote for resistance to stripe rust or to leaf rust, respectively, by which these lines were first selected.
**IT values were converted into 1 (resistant) to 9 (susceptible) scale as follows: IT = 1 (0/0; /0; −l−/1− −1/1−1+), IT = 3 (1+/1−2/2), IT = 5 (2−2+), IT = 7 (2−3−/2, 3−/2, 3), IT = 9 (3−/3/3+). Numbers in parenthesis indicate scores determined based on visual evaluation of fungal coverage and sporulation.
***Reflects apparent heterozygous secondary recombinant 6B chromosome

Pathogens

Leaf rust isolate #526-24 and stripe rust isolate #5006 from the stocks of the Institute for Cereal Crops Improvement were used. The virulence/avirulence (V/Av) formulae of these isolates are Lr1,3,24,26,10,18,21,23,15/Lr2a,2c,9,16,3ka,11,17,30 and Yr6,7,8,9,11,12,17,19,sk,18,A/Yr1,5,10,15,24,26,sp, for isolate #526-24 and #5006, respectively. Both of these isolates were used to select resistant progenies at seedling stage and to evaluate adult plant resistance. Both isolates are virulent to Galil and represent highly virulent pathogen races.

Inoculation and Disease Evaluation

Seedling Stage

Seedlings of each generation were tested and selected for sensitivity to leaf and/or stripe rust. Plants were grown in small pots in a temperature-controlled greenhouse at 22±2° C. Seven to 10 days-old seedlings were inoculated by spraying to runoff with about 1 mg of urediniospores suspended in 800 μl of lightweight mineral oil Soltrol® 170Isoparaffin (ChevronPhillips). After evaporation of the oil, the leaf rust-inoculated plants were maintained in a dew chamber at 18° C. for 24 h and then moved to a greenhouse. Stripe rust-inoculated plants were maintained in a dew chamber having a temperature of 9° C. for 16 h in the dark followed by 15° C. in light and then moved to a growth chamber having a temperature of 15° C. and 12 h light/12 h dark regime. Symptoms were scored 10-12 days post inoculation for infection type (IT) on a standard 0-4 scale. ITs of 0-2 were considered indicative of a resistant response and 3-4 as a susceptible response.

Adult Plants

Leaf Rust (Plant Grown in Greenhouse)

Single homozygous BC₄F₄ primary- and BC₂F₄ secondary-recombinant plants were grown in 5 L pots placed in a cooled greenhouse and sprayed with urodiniospores at the seventh leaf stage (stem elongation, stage 3 - Zadoks scale, Zadoks J C et al. 1974. Weed Research 14: 415-421). Four groups of plants, each containing two pots for each genotype, were inoculated similarly to the seedling inoculation. Disease level was evaluated after spike emergence, about 3 weeks post inoculation.

Stripe Rust (Plant Grown in the Field)

Seeds of the same primary and secondary recombinants that were used in seedling assays were planted in nursery plots under field conditions. Each plot (single genotype) consisted of four 1m rows 20 cm apart and a single margin row 40 cm apart of the spreader cv. Falchetto. The Falchetto plants were inoculated with urediniospores six times between January 14 (when the Falchetto plants were at seventh leaf stage) to February 5, by either brushing the upper leaves with a bundle of highly infected leaves, or by dusting the plants with a mixture of urediniospores and talc using a manual air pump. High humidity and cool nights prevailed in the experimental area during this period.

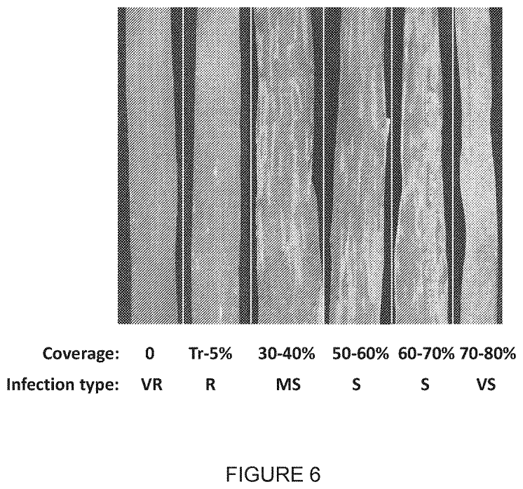

Disease level was measured with each nursery plot being evaluated as a whole unit, by % of rust coverage and general reaction (Susceptible—Intermediate— Resistant). An example of reaction is demonstrated in FIG. 6.

Shortening the Alien Segment

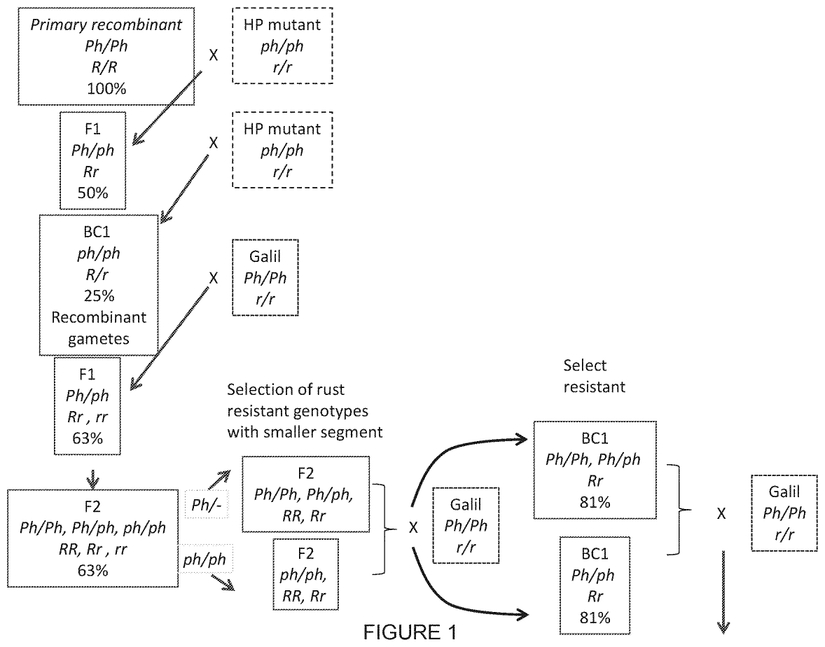

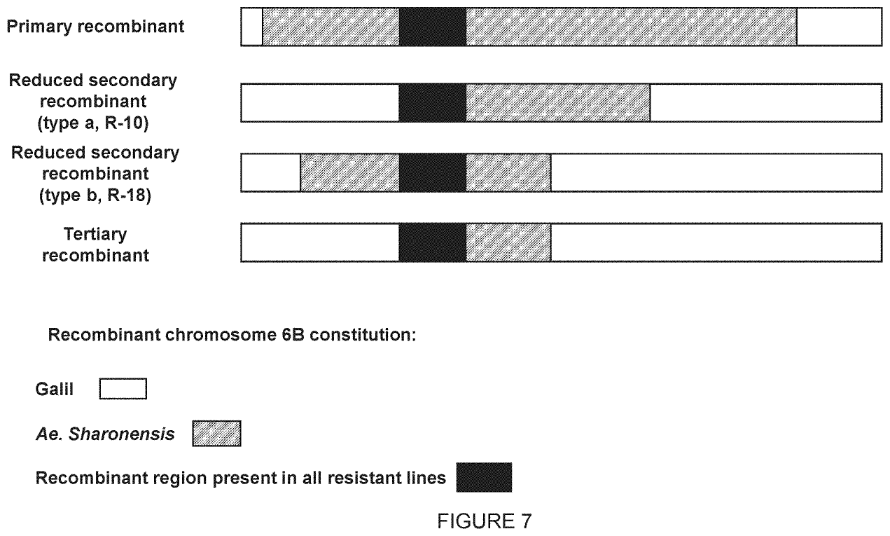

Secondary recombinants (SR) with shortened alien segment were obtained by induction of homoeologous recombination in hybrids between the primary recombinants and the wheat cv. Galil, followed by phenotyping and molecular selection as depicted in FIG. 1 and FIG. 7. Briefly, selected primary recombinants with different sizes of alien introgression on chromosome 6B (Table 2) were pollinated by the HP mutant and the F₁ offspring were backcrossed to the HP mutant. Rust resistant plants that were homozygous for ph1b were selected and pollinated by Galil. The resulting hybrids, were screened for reaction to both pathogens and 6B chromosome constitution of resistant plants was determined using molecular markers (using PCR markers as described in Table 3 and Example 1 hereinbelow). Plants with reduced size of the alien segment compared to the fragment size in parental primary recombinants were selected and allowed to self-pollinate. Selected rust resistant F₂ plants were characterized for their Ph1 genotype. Ph1/− were considered secondary recombinants, while homozygous ph1b were allowed for another recombination event. Both groups of plants were pollinated by Galil to produce BC₁ progeny. Chromosome 6B of BC₁ plants of the latter group was molecularly analyzed with the PCR markers and one tertiary recombinant was obtained. All of the rust resistant BC₁ recombinants were backcrossed again to Galil. BC₂ plants that were resistant to the leaf and stripe rust isolates were self-pollinated. Resistant BC₂F₂ progeny of each recombinant that are also homozygous for the SR 6B chromosome were selected as described hereinbelow. BC₂F₃ seeds of these plants were pooled and used for seed propagation in the greenhouse.

Further Shortening of the Segment

The recombinant line R-10, in which the alien chromatin extended towards the telomere of the long chromosome arm (right extension; FIG. 7, type a) and line R-18 in which the alien chromatin extended towards the telomere of the short chromosome arm (left extension; FIG. 7, type b) were crossed aiming to curtail the extensions towards the telomeres while maintaining the alien region around the resistance (FIG. 7, tertiary recombinant). The hybrids were pollinated by Galil and their desired offspring were selected using the PCR markers (Table 3).

Molecular Characterization of the Alien Segment

DNA Extraction

Leaves were collected and stored at −80° C. Frozen leaf samples (50 mg) were freeze-dried using Lyophilizer (Blue Wave, BW-10-ORD) for 16 h, and grinded for 1 min at 1,500 rpm, using two ⅛″ and one 3/16″ stainless steel beads in a Tissue-lyser (GenoGrinder). DNA was extracted using E-Z 96 Plant DNA Kit (Omega) (for PCR analysis), or using DNeasy Plant mini kit (Qiagen) (for GBS), according to manufacturer instructions.

Selection of ph1b Mutants

The presence of the Ph1 allele was detected by marker PSR2120, amplified by a forward primer having SEQ ID NO:19 and reverse primer having SEQ ID NO:20 (Table 3) according to Qu et al. (Qu L I et al. 1998. Theor Appl Genet 96:371-375). Plants deficient for the corresponding band were considered as ph1b/ph1b mutants.

Genotyping by Sequencing (GBS)

GBS was performed on 100 samples comprised of cv. Galil, the HP mutant, and 74 resistant and 24 susceptible secondary recombinant F₁ plants (FIG. 1), most of which were derived from the primary recombinant line RY 32-3-14 (Table 2). DNA was isolated from leaves of young plants (one month old) as described above. Sequencing was performed according to a modified Restriction site Associated DNA Sequencing (RAD-Seq) method (Elshire R J et al. 2011. PLoS One 6, e19379) at AgriLife Genomics (Texas). Briefly, genomic DNA was digested with the restriction enzyme PstI and approximately 110 bp were sequenced from both sides of the fragments. For sequencing, single end V4 chemistry with 125 bp kit were used on Illumuna Hiseq 2500 resulting in 40 M reads per sample of Galil and the HP mutant, and 8 M-10 M reads per sample for the rest of the recombinants. Raw data were analyzed by NRGene (nrgene.com/, Israel), using the newly assembled wild emmer ‘Zavitan’ genome (Avni R et al. 2017. Science 357:93-97) as a reference.

Development of Molecular Markers for the Alien Segment

According to the frequency of recombination events, single nucleotide polymorphisms (SNPs) between Ae. sharonensis and wheat sequences were mapped along the introgression region. Based on the SNPs, nine PCR primers were designed following the instructions mentioned at Ayyadevara et al. (Ayyadevara S et al. 2000. Anal Biochem 284:11-18). The primers for the marker amplification are described in Table 3 hereinbelow. An online available database was used to check for repetitive elements within the markers (wheat.pw.usda.gov/ITMI/Repeats/blastrepeats3.html).

Conversion from the wild emmer map sequence to CS map sequence and SNPs detection was accomplished as follows: raw reads were de-multiplexed using GBSX (Herten K et al. 2015. BMC bioinformatics 16:73). Reads of each genotype were aligned to chromosome 6B of the CS reference genome using Burrows-Wheeler Aligner (BWA) (Li H and Durbin R. 2009. Bioinformatics 25:1754-1760). Sequence Alignment/Map (SAM) tools (Li H. 2011. Bioinformatics 27:2987-2993) were used to pileup the individual alignment files into one pileup file that was used by BCFtools CALL (samtools.github.io/bcftools/) to call the SNPs. VariantAnnotation R package (Obenchain V et al. 2014. Bioinformatics 30:2076-2078) was used to read the SNP data into R environment. For each SNP a Fisher Exact test was conducted using a 2×2 contingency table of resistant and susceptible genotypes against the origin of the SNP allele (CS as “reference” SNP or the alien “alternative”). -log P-values of the Fisher test were plotted against the position of the corresponding SNP. Higher -log P-values (i.e. lower P-values) indicated the probability of the SNPs-resistance association to be non-random. SNPs with -log P>16, were assumed to be associated with the resistance locus.

Table 3 hereinbelow presents PCR markers for mapping the Ae. sharonensis alien segment. In all markers, which are based on SNPs, the polymorphic nucleotides are marked in bold and underline. Markers 1-7 were used to characterize the length of the segment. Markers 8-9 were used for detection of the critical area for the resistance. Marker 10 was used for selection of ph1b/ph1b lines. Markers 11-12 were used simultaneously for selection of plants homozygous for the segment (plants in which marker 11 was present and marker 12 was absent were selected). Temperature cycling consisted of 95° C. for 5 min, followed by 32 cycles of 95° C. for 30 sec; annealing at a temperature depending on the Taq enzyme used for 30 sec; 72° C. for 30 sec; and a final extension step at 72° C. for 5 min.

TABLE 3
PCR markers

| | | | | Marker | |
|---|---|---|---|---|---|
| Marker | Marker | Primer (5′-3′)* | | size | |
| No. | Name | Forward | Reverse | (bp) | Marker Sequence |
| 1 | 2-3HS2 | CAAACACCACAACAG | CAGCCCGAAGGAAAACA | 154 | CAAACACCACAACAGCTATGTCTTCAACTCGCTGCAG |
| | | CTATG | AT | | CCCTCTGCTTCACCCCCGCTACAGTCTATCCAGGAAG |
| | | (SEQ ID NO: 32) | (SEQ ID NO: 33) | | ATGCGCGCCTCTGGGGTAGGGCAAGAGCCAAAGGAAT |
| | | | | | GAATATGCTTCCGAGAGTTAGGCCATTGTTTTCCTTC |
| | | | | | GGGCTG |
| | | | | | (SEQ ID NO: 34) |
| 2 | 2-3HS3 | CAATTGGCATAAGAG | CAATTGGCATAAGAGCC | 144 | CAATTGGCATAAGAGCCTTGTCGATCCGAGGCGACTG |
| | | CCTT | TT | | TGGCGGTGCGGCGGACTCCATGTGAAGGGGACGTCAG |
| | | (SEQ ID NO: 37) | (SEQ ID NO: 38) | | CGGCGAAAGGTGGACGCCGTTGGCGGCGCGATGCTGC |
| | | | | | TAGCGGTGGGTGTGTGTCTTCATCATCGTCGAG |
| | | | | | (SEQ ID NO: 39) |
| 3 | 1C | GGCCAGTGCAATAAA | ATTTGTAGTAAGAGTGC | 214 | GGCCAGTGCAATAAACTAATAACACAAATCTAATTAG |
| | | CT | G | | TGCCAAAACTAGAACGAAATAAAATTATCTTTGTGCA |
| | | (SEQ ID NO: 40) | (SEQ ID NO: 41) | | TGCCCTCATTGCCGTGAAAGGGGCAGCAAATTAGCTG |
| | | | | | CAGTATCTCATGCGTACACCATGTTCTTTGTAGTAAA |
| | | | | | TCAGGAAGAAACATGCAAAACCCACGAATTACTCAAC |
| | | | | | ACTAAGCATGCCGCACTCTTACTACAAAT |
| | | | | | (SEQ ID NO: 42) |
| 4 | 2S1 | AAAAGAAAGTTGGCC | CGGCATGATTAAAACAT | 128 | AAAAGAAAGTTGGCCCCGAAATGTAACTGCATGCAGG |
| | | CCG | GAGGCAA | | ATGTATGGTTTATGATGTTGATGAAGGATGGAATACA |
| | | (SEQ ID NO: 43) | (SEQ ID NO: 44) | | CCACACCATGGGGGGCGATGTTTGAGAGCCTTGCCTC |
| | | | | | ATGTTTTAATCATGCCG |
| | | | | | (SEQ ID NO: 45) |
| 5 | 2S2 | TCATCGATGGGATCG | ATGTCCACCTGTCCCAA | 209 | TCATCGATGGGATCGACCATGAGGTGCAAAGGGAAGA |
| | | AC | G | | AGAGACTGGATGCTTCAGGCTATGGGTATGGATGGAT |
| | | (SEQ ID NO: 46) | (SEQ ID NO: 47) | | GCAGTACAGAAGCTCAAGACGAGAGGCGTGCTGCATG |
| | | | | | CAGCTTGAGGAGCCACGGGAGAGGGGCTCGCCCGGGA |
| | | | | | TGCATTTCCTTGAGCTCAACATCCTCAAGGAGGTGCC |
| | | | | | GCAACGCTTGGGACAGGTGGACAT |
| | | | | | (SEQ ID NO: 48) |
| 6 | 3S1 | ATCCTATCGCTCAAC | ACAGTTAGCTTGGCTTC | 147 | ATCCTATCGCTCAACATGTTCTGGTTCACTTTTTTAG |
| | | ATG | A | | ATGAAGATGGGAATAACCTGCTTAACATCTTCTGGTT |
| | | (SEQ ID NO: 49) | (SEQ ID NO: 50) | | CACTTTTGTAGATGAATATGGGAATAACCTGCATGCA |
| | | | | | GTCACGGGAATGGAGTGATGAAGCCAAGCTAACTGT |
| | | | | | (SEQ ID NO: 51) |
| 8 | 3S4 | GCTGCGTAAAATTAA | CTTTTAGTCAATTCTTG | 180 | GCTGCGTAAAATTAAGCATTATGAACAAGCAATCAGG |
| | | GCA | GTC | | CGGATTTTTAGGGATAATTCACTAATGTGGTTAACTT |
| | | (SEQ ID NO: 52) | (SEQ ID NO: 53) | | ATTTCTAGTAATGTAGAAGGTTCAACTTTTGACTGCA |
| | | | | | TGCAGAACCAAATAGCTCTAGGTGACAGACCAGAGAT |
| | | | | | CCCATAAGTTACGACCAAGAATTGACTAAAAG |
| | | | | | (SEQ ID NO: 54) |
| 9 | 4C | CTCAATCATTTCCGT | CTACGCAACAAGGAAAA | 191 | CTCAATCATTTCCGTCTACTCCTACGCAATCGTGCGA |
| | | CTAC | CA | | CGCTTCTGAAGAATATGAAAAATATGGTGTGGATCTT |
| | | (SEQ ID NO: 55) | (SEQ ID NO: 56) | | CTGAAATCCGAAGCTTCACTGCAGTACTTATGCAACC |
| | | | | | TACCACCTCATAGGTCTGCTTTACGTTACTAAAATTG |
| | | | | | GCGAAGACTTGTGATTCTGCATTGTGTTTTCCTTGTT |
| | | | | | GCGTAG |
| | | | | | (SEQ ID NO: 57) |
| 10 | PSR2120 | TTAACGCCAGGGCAT | CTGCAGGAGGCGCTGGA | 232 | Qu et al. (1998) |
| | | ACTC | | | |
| | | (SEQ ID NO: 58) | (SEQ ID NO: 59) | | |
| 11 | Zyg_1Sh_1 | CAAACACCACAACAG | CAGCCCGAAGGAAAACA | 154 | SEQ ID NO: 34 |
| | | CTATG | AT | | |
| | | (SEQ ID NO: 32) | (SEQ ID NO: 33) | | |
| 12 | Zyg_2G_2 | CAATTGGCATAAGAG | CATAGCCATCACCACCT | 219 | CAATTGGCATAAGAGCCTTGTCGATCCGAGGCGACTG |
| | | CCTG | TG | | TGGCGGTGCGGCGGACTCCATGTGAAGGGGACGTCAG |
| | | (SEQ ID NO: 60) | (SEQ ID NO: 61) | | CGGCGAAAGGTGGACGCCGTTGGCGGCGCGATGCTGC |
| | | | | | TAGCGGTGGGTGTGTGTCTTCATCATCGTCGAGCTGC |
| | | | | | ATCACGAGCGATGGAGAGAGATGTGCTGCTCGGTAAG |
| | | | | | TTGTGGCACCGGCGGCAAGGTGGTGATGGCTATG |
| | | | | | SEQ ID NO: 62 |

### Example 1: Production of the Secondary Recombinants with a Shorter Alien Segment

Primary recombinant lines were hybridized and backcrossed with Sears' high pairing mutant (ph/b, HP mutant). Homozygous ph1b progenies that were heterozygous for the wheat and its homoeologous alien segments were selected and crossed with the elite spring wheat cv. Galil. A population of 1240 plants of this progeny was inoculated with the leaf rust and stripe rust isolates described hereinabove. A total of 594 plants were found resistant to both isolates, 45 were found resistant to one of the isolates (28 to leaf rust and 17 to stripe rust), and the other 601 plants were susceptible to both isolates.

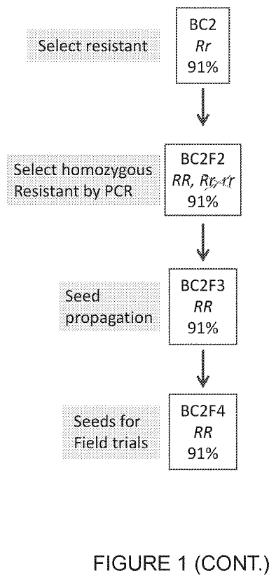

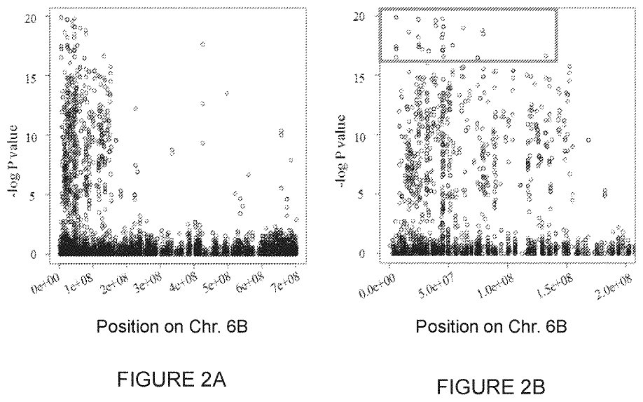

DNA from 100 resistant and susceptible progenies (including cv. Galil, the HP mutant, and 74 resistant and 24 susceptible secondary recombinant F₁ plants) was sequenced and analyzed as described hereinabove. A genetic map of chromosome 6B was generated based on frequency of recombination events and using the genome sequence of wild emmer wheat as reference. The GBS analyses revealed SNPs scattered along the homoeologous recombination region on chromosome 6B. In order to define more precisely the location of the alien segment in specific introgression lines, the sequences of the recombinants were re-aligned to the sequence of the bread wheat cultivar Chinese Spring (CS) (IWGSC RefSeq v1.0, wheat-urgi.versailles.inra.fr/Seq-Repository/Assemblies). Fisher Exact test was conducted to discover non-random SNPs associated with resistance. Totally, 1,362 SNPs were found, most of which were located in the range of about 0-160 Mbp (FIG. 2A). Twenty-six of these SNPs had P values of -logP>16 (FIG. 2B) and were the most likely to distinguish between the chromatin of Ae. sharonensis and cv. Galil. The recombined area was further divided into four regions, according to the frequency of recombination as reflected by the SNPs.

Seven of these SNPs, spanning the entire segment, were used to develop nine PCR probes, which distinguished between the Ae. sharonensis and wheat along this region (Table 2). The PCR probes were used to screen 520 secondary recombinants resistant to both stripe rust and leaf rust isolates.

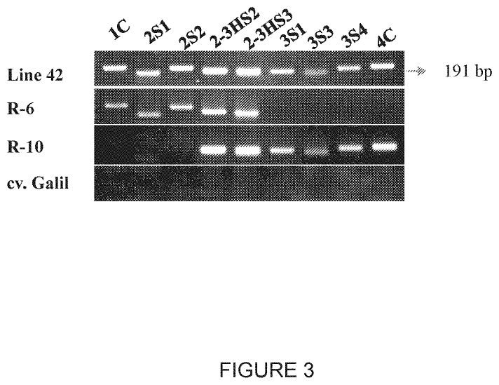

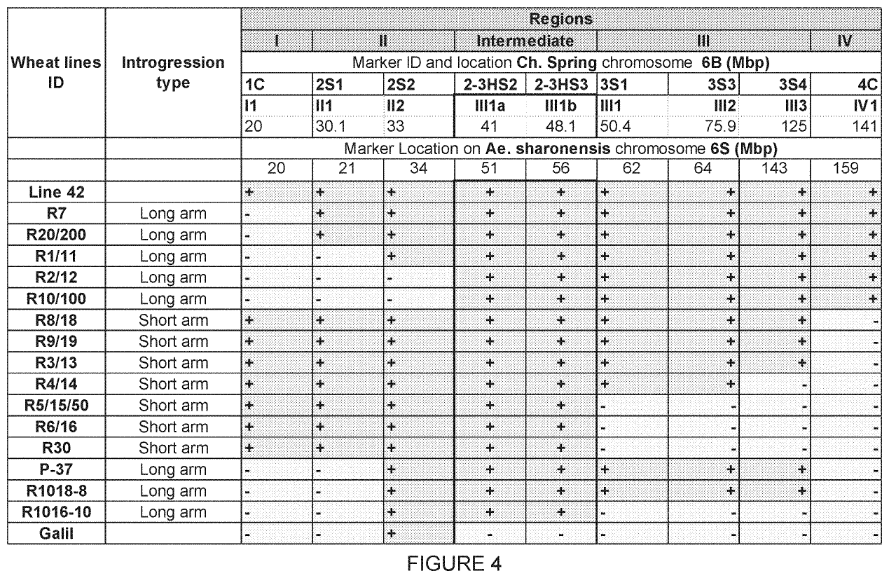

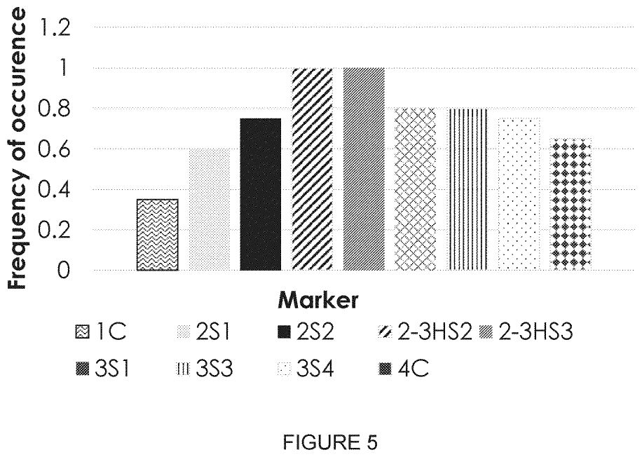

At first, all the plants were screened with the two distal markers designated 1C and 4C (Table 2). Presence of both markers indicated lack of recombination within the alien segment, absence of one of the markers indicated recombination on either the left side (lack of marker 1C), or the right side (lack of marker 4C) of the alien segment. 38 plants deficient for one of the markers were identified, and these plants were further evaluated using the remaining five PCR markers. Based on the PCR analyses a set of 20 secondary recombinant (SR) plants was selected. These plants were re-evaluated for resistance and screened again with the PCR markers (FIG. 3). From these plants, a final set of 13 SR lines was selected based on the length of the alien fragment, the introgression pattern (FIG. 4), and phenotypic resemblance of the plants to cv. Galil. All of these lines contained a common region of 17.4 Mb that was detected between markers 2S2 and 3S1, regardless if the break point of the recombination occurred at the right or the left end. To verify this region, two additional markers, 2-3HS2 and 2-3HS3 (Table 2) were developed and used to analyze the selected recombinants as well as randomly selected susceptible lines as a negative control. Both markers were present in all of the resistant recombinants (FIG. 4) and absent in the susceptible lines. The frequency of occurrence of each marker was calculated to indicate the rate of recombination along the chromosome segment (FIG. 5). Markers 2-3HS2 and 2-3HS3 were both present in all resistant plants, 3S1 and 3S3 were each present in 16 plants, 2S2 and 3S4 were each present in 15 plants, 4C in 13 plants, 2S1 in 12 plants, and 1C was present in seven plants. In total, 2.5% (13/520) and 1.34% (7/520) of the resistant plants had a secondary recombination on the left end (indicated by lack of marker 1C) or the right end (indicated by lack of marker 4C) of the introgression segment, respectively.

The 13 selected SR lines were allowed to self-pollinate and between 20-30 F₂ offspring of each SR line were evaluated for their reaction to the leaf and stipe rust isolates. PCR analysis of the resistant F₂ plants with Ph1 specific primers revealed 12 plants that contained the Ph1 allele and one plant lacking the allele. The 12 Ph1 -poitive plants had different alien chromatin constitution: in seven plants the alien segment extended towards the short arm telomere, while in the remaining 5 plants the alien segment extended towards the long arm telomere (FIG. 4). All of these SR 6B plants were backcrossed to cv. Galil and rust resistant (R/r) progenies were selected. In this cross, the single genotype that was found homozygous for ph1b underwent another homoeologous pairing event and produced a tertiary 6B recombinant (TR) chromosome in line P-37 (FIG. 4). All of the 12 SR lines and the single TR line were further backcrossed (BC2) to cv. Galil (FIG. 1) in order to recover the cv. Galil genetic background.

### Example 2: Identifying Shorter Resistance-Conferring Segment

To further reduce the size of the alien segment, a cross between two SR BC₂ plants was performed, one with a right arm tail and one with a left arm tail (FIG. 7). The progeny of this cross was analyzed by PCR using the diagnostic primers (Table 2) and two hybrids were selected. One hybrid (R-1018) contained an alien segment that was derived from chromosome 6B in SR lines R-18 and R-10 and contained the 2-3HS2 and 2-3HS3 markers (FIG. 4). The alien segment in this line was limited by markers 2-3HS2 and 3S4, indicating even a shorter segment than in the TR line P-37 (FIG. 4). The other hybrid (R-1610) contained an alien segment that was derived from chromosome 6B in SR lines R-16 and R-10 and contained the 2-3HS2 and 2-3HS3 markers but also was limited by these markers. This hybrid possessed the shortest segment of all the introgression lines. Unexpectedly, self-pollination of the heterozygous hybrid (comprising one recombinant and one naïve chromosome) resulted in a complete heterozygous progeny.

### Example 3: Selection of Homozygous Plants

Homozygous resistant plants were selected from self-pollinated SR BC₂ progeny by their alien segment status as follows: At least 100 BC2F2 plants from each SR line were screened with the PCR markers Zyg_1_Sh_1 and Zyg_2 G_2 (Table 2). The marker Zyg_1Sh_1 (1Sh_1) detects SNP of Ae. sharonensis. This marker amplifies the same sequence as 2-3HS2 from the same middle region; however, 1Sh_1 is always used in pair with Zyg_2 G_2 (2G_2), which detects the SNP of Galil, such that in homozygous plant for Ae. sharonensis using 1Sh 1 will result in an amplified band, and using 2G_2 will not. In heterozygous plant, both primers will give a band. Self-fertile lines with cv. Galil morphology, that had the desired alien chromatin and lacked the cv. Galil PCR marker were selected. BC2F3 seeds of the selected homozygotes of each SR line were pooled and used to produce BC2F4 seeds for further experiments including resistance to stripe rust in the field.

### Example 4: Resistance Validation

Leaf Rust

Resistance of adult plants of representative SR line plants (of the SR lines presented in FIG. 4) was examined as described hereinabove. The reaction of the recombinant lines is presented in Table 4 as percentage of uredinia coverage and infection type (IT) value. The latter was converted into 1 to 9 score scale as specified in the legend of Table 4. A score of 9 was considered highly susceptible whereas scores of 1 to 7 reflected descending values of resistance. All the recombinant lines were highly resistant except line R-4 which segregated into highly resistant or susceptible individuals. Wheat cv. Galil had more than 80% coverage and was scored 9 IT value, hence considered as a susceptible genotype.

TABLE 4
Reaction of adult SR lines to inoculation with leaf rust isolate #526-24 in the greenhouse

| | | Flag leaf | | −3 leaf | | Overall |
|---|---|---|---|---|---|---|
| Line | | % coverage | IT value | % coverage | IT value | plant reaction |
| R-1-2-103 | | 7.4 | 1(3) | 26.0 | 1 | R |
| R-1-2-104 | | 3.4 | 1 | 19.1 | 1(3) | R |
| R-2 | | 25.0 | 1-3(7) | 32.2 | 1(3) | MR |
| R-3 | | 2.8 | 1 | 14.3 | 1 | R |
| R4 | R | 7.0 | 1 | 20.0 | 1 | R |
| | S | 70.0 | 9 | 80.0 | 9 | S |

| | | | | | |
|---|---|---|---|---|---|
| R-16 | 6.2 | 1 | 21.0 | 1(3) | R |
| R-7 | 10.0 | 1 | 24.5 | 1 | R |
| R-18 | 1.8 | 1 | 10.1 | 1 | R |
| R-19 | 6.7 | 1(3) | 16.9 | 1 | R |
| R-10 | 1.8 | 1 | 20.6 | 1 | R |
| R-20 | 8.2 | 1(3) | 18.9 | 1 | R |
| P-37 | 0.8 | 1 | 16.6 | 1 | R |
| Line-33 | 2.3 | 1 | 14.1 | 1 | R |
| Line-34 | 3.4 | 1 | 10.6 | 1 | R |
| Line-42 | 3.4 | 1 | 16.3 | 1 | R |
| Galil | 81.7 | 9 | 63.8 | 9(7) | S |

Table 4: Values are mean of 10 plants. IT values in brackets denote rare scores on the same leaf. IT values were converted into 1 to 9 scale as follows: IT=1 (0/0; /0;-1-/1 - -1/1-1+), IT=3 (1+/1-2/2), IT=5 (2-2+), IT=7 (2-3-/2, 3-/2,3), IT=9 (3-/3/3+). Line R-4 segregated into half R and half S plants. R-resistant; MR-moderately resistant; S-susceptible

Stripe Rust

Resistance to stripe rust was examined as described hereinabove, using strip rust isolate #5006. All the recombinant lines were also highly resistant (very resistant, VR) with 0 coverage (R-1-2-103 and R-1-2-104 gained 0-traces) (FIG. 6), except R-4 which segregated into 0 (very resistance, VR) and 50-60% coverage designated susceptible (S). The Galil variety scored 30-40% as medium susceptible (MS) to susceptible (S) and additional variety, Falchetto, which was planted in every plot, scored 60-70% (S) or 70-80% as very susceptible (VS).

### Example 5: Analysis of the Resistance-Conferring Segment

MutChromSeq is an approach for isolation of genes and DNA sequences controlling gene expression in plants with complex and polyploid genomes. It is a lossless complexity reduction based on flow sorting and DNA sequencing of mutant isolated chromosomes. Comparison of sequences from wild-type parental chromosome with chromosomes from multiple independently derived mutants identifies causative mutations in a single candidate gene or a noncoding sequence (Steuernagel B. et al. “Rapid Gene Isolation Using MutChromSeq.” Wheat Rust Diseases. Humana Press, New York, N.Y., 2017. pp. 231-243).

Dataset.

EMS mutagenesis was performed on resistant primary introgression plants (Line 42 and 34), according to Sanchez-Martin et al., 2016 (Genome Biology 17(1):221). Briefly, seeds of the resistant recombinant lines were treated with EMS (M1 population), and grown to obtain M2 generation. Screens for loss-of-resistance to leaf rust isolate #526-24 and stripe rust isolate #5006 were performed on 1,396 families and 16 susceptible mutants were identified (Table 5).

TABLE 5
Resistant lines mutated to be susceptible

| | | Submitted | |
|---|---|---|---|
| Resistant | Susceptible | to | Submitted |
| Parent | mutants | chromosome | to |
| designation | designation | sorting | Sequencing |
| Line 42 | 10.6 | + | + |
| | 17.1 | + | |
| | 20.2 | + | + |
| | 28.1 | | |
| | 57.2 | | |
| | 69.4 | | |
| | 78-9 | + | + |
| | 109.2 | + | + |
| | 125.5 | + | + |
| | 134.3 | + | + |
| Line 34 | 2.2 | + | |
| | 8.1 | | |
| | 9.1 | | |
| | 21.1 | + | + |
| | 23 | | |
| | 25.2 | | |

All of the mutants were further validated in M4 generation and crossed with the susceptible Galil and resistant plants to confirm that the mutation was in the gene candidate and the monogenic nature of the gene candidates. Nine susceptible mutants were chosen for the 6B chromosomal sorting and 7 final mutants underwent sequencing. Six mutants that provided acceptable sequence data were used for scaffold alignment (mut_10.6, mut_20.2, mut_78-9, mut_109.2, mut_125.5 and mut_134.3). One of the mutants (mut_10.6) seemed to have more heterozygosity than the rest of the mutants. Due to the strict filtering among the mapped reads and by comparing to other mutants, these heterozygous SNPs look as true ones and not a product of unspecific read alignments. Some of the candidate scaffolds were found to be heterozygous for SNP mutations in mut_10.6. This fact was taken in account when evaluating the scaffold candidates.

Methods.

Read Quality Control

Sequenced libraries QC was assessed with fastqc. All reads from wild type (wt) and mutants were of good quality, and thus no trimming was required.

Masking for Repetitive Elements

The Line 42 (wt=R) and Ae. sharonensis reference assemblies were masked for repetitive elements using RepeatMasker and the Triticeae Repeat Database as the source for repeats.

MutChromSeq on Line 42 (Wt) and Mutants

The alignment was performed according to the approach outlined by Sanchez-Martin et al. (2016, ibid). with some modifications. The initial protocol produced many unspecific alignments, likely due to the contaminations from other chromosomes, and this made the screening for SNPs difficult in many scaffolds. Particularly, mem aligner, instead of the aln aligner of the original protocol was used. In addition, the filtering of mapped reads after alignments was strict, enabling to eliminate unspecific alignments. The drawback of this approach is a lower read depth and the chance to miss some true SNPs. Later visualization of candidate scaffolds helped alleviating this issue. To identify SNPs, the results retrieved with the different aligners and strict/relaxed parameters were used, as well as visual inspection.

MutChromSeq on chr6S Fragments

The Line 42 (wt) assembly has 91,266 scaffolds and a median contig size of the genomic assembly (N50) of 12,647 and with scaffolds as small as 445 bp. In a worst-case scenario, the gene of interest could be split between two scaffolds. This would make the gene undetectable by MutChromSeq. To minimize this possibility, a “pseudo” MutChromSeq approach on the entire 6S chromosome was taken. First, the wheat recombinant chromosome 6B (r6B; wheat chromosome 6B with introgression from Ae. sharonensis chromosome 6Ss^{h}) which underwent mutagenesis was divided into fragments of 25, 50, 75, and 100 kb and MutChromSeq was performed for these fragments (4 times) as if they were assembled scaffolds. This approach produced a greater number of SNPs since the reference and the mutant origin from different genetic background. Nevertheless, detecting the mutations produced by EMS and distinguish those from the polymorphisms between different genetic backgrounds may be done by eliminating SNPs that are common to all mutants and wt, and only considering unique SNPs. This “pseudo” MutChromSeq approach also provides an extra level of validation: the candidate scaffolds from the standard MutChromSeq should map to the genomic positions retrieved by the “pseudo” MutChromSeq on the chromosome 6S^{sh} fragments.

Scaffold Visualization

Candidate scaffolds were individually visualized with the Integrative Genomics Viewer (IGV) to validate or discard putative SNPs. To have a better picture of candidates, aligned reads from the results of using different aligners and with different parameters were used in this step.

Alignment of Scaffolds to Reference

Gmap and blastn were used to assess whether a scaffold contains wheat or Ae. sharonensis DNA. Chinese Spring wheat genome reference assembly (IWGSC RefSeq v1.0) chromosome 6B, and the Ae_sharonensis_v1 chromosome 6S^{sh} assembly provided by Brande Wulff (unpublished data), were used as references. Gmap is a better aligner for this purpose since it can find full length, global alignments, while blast usually retrieves more fragmented, local alignments. As a consequence, with the parameters used (min. coverage 90% and min. identity 90%), Gmap sometimes did not produce any hit. There are two main reasons for this to happen, first, the scaffold is an artifact, and second the reference genome does not contain the sequence of the scaffold. The latter was considered to be more likely to happen for the Ae. sharonensis genome since the wheat reference is a more curated one. Thus, if gmap does not retrieve a hit, the scaffold is more likely to be from Ac. sharonensis. A good candidate scaffold should also map in or near the 33-50.4 Mb window that was highlighted using GBS markers.

Results

Since only six mutants had acceptable sequence data, and due to lack of a clear candidate while examining scaffolds harboring SNPs in 6 or 7 mutants, scaffolds with SNPs in 4 or more mutants were selected for further inspection, as long as these scaffolds mapped to Ae sharonensis chromosome 6S^{sh} and not to chromosome 6B of Chinese Spring wheat. A good candidate scaffold was also defined as having the SNPs most likely produced by EMS, that is G/C to A/T. Ideally, these SNPs should be spread within a genomic distance of no more than ˜10 kb, when considering all mutants. Finally, a candidate scaffold should map to the 33-50.4 Mb genomic window in chromosome 6B of CS.

Candidates that answered the above-described requirements are summarized in Table 6 hereinbelow. Of these candidates, scaffolds 19799 mapped around 57.5 Mbp; scaffold 00757 mapped at around 57.4 Mbp; scaffold 1934717 mapped around 57.7 Mbp, scaffold 1548600 mapped around 56.7 and scaffold 20860 mapped around 44 Mbp on Ae. sharonensis chromosome 6Ss^{h}, are of interest. These scaffolds are associated with marker 2-3HS2 and are located in the genomic region of 34-62 Mbp on the 6S^{sh} of Ae. sharonensis (minimal region that is present in all of the resistant lines as defined by PCR markers 1-9 (Table 3), shown to be linked to the rust resistance. In addition, these scaffolds contain SNPs in or close to potential resistance genes.

TABLE 6
Data of selected scaffolds

| | | | | | | | Average | |
|---|---|---|---|---|---|---|---|---|
| | | | | | | | No. of | |
| | | No. of | | | | | SNPs | |
| | | mutants | Position | Position | SNP | No. of | within | |
| Scaffold | Length | with | on CS | on AES | scattering | annotated | single | |
| Name | (bp) | SNPs | (Mbp) | (Mbp) | (kb) | ORFs | ORF | Comments |
| 101631 | 19950 | 6 | 51 | 13 | 16 | 4 | 2 | |
| 36508 | 64552 | 6 | 21 | 18 | 50 | 1 | 0 | Out of |
| | | | | | | | | window |
| 145403 | 39889 | 6 | 28 | 25 | 23 | 3 | 0 | Out of |
| | | | | | | | | window |
| 68474 | 11393 | 6 | 26 | 29 | 7 | 2 | 1 | Out of |
| | | | | | | | | window |
| 1540406 | 64196 | 5 | 31 | 32 | 13 | 1 | 0 | |
| 1900261 | 24799 | 5 | 33 | 33 | 13 | 3 | 0 | |
| 1933170 | 29906 | 5 | * | 34 | 9 | 1 | 0 | |
| 1531163 | 16584 | 5 | * | 35 | 7 | 2 | 1 | |
| 1927703 | 61472 | 6 | 34 | 35 | 30 | 1 | 2 | |
| 1895355 | 16716 | 3 | 52 | 38 | 8 | 2 | 2 | |
| 1931208 | 45874 | 6 | 35 | 38 | 27 | 2 | 2 | |
| 1935984 | 100249 | 5 | 39 | 39 | 30 | 2 | 1 | |
| 1913977 | 35574 | 5 | * | 42 | 22 | 4 | 0 | |
| 1916860 | 13004 | 5 | * | 43 | 7 | 1 | 1 | |
| 20860 | 19882 | 5 | 41 | 44 | 13 | 3 | 2 | Preferred |
| | | | | | | | | candidate |
| 1891763 | 32429 | 6 | 44 | 44 | 28 | 4 | 0 | |
| 1937448 | 32664 | 5 | 41 | 44 | 23 | 2 | 1 | |
| 1896571 | 26451 | 4 | * | 46 | 20 | 1 | 1 | |
| 1528020 | 22991 | 5 | 42 | 47 | 15 | 1 | 0 | |
| 1935712 | 23698 | 4 | 45 | 47 | 7 | 1 | 2 | |
| 1893110 | 27610 | 4 | 48.5 | 48 | 23 | 1 | 0 | |
| 143060 | 19845 | 4 | 41.8 | * | 9 | 0 | 0 | |
| 1898148 | 53213 | 5 | 52 | 51 | 20 | 1 | 1 | |
| 1934541 | 75783 | 6 | 51 | 51 | 60 | 1 | 1 | |
| 1538547 | 15026 | 6 | 56 | 56 | 6 | 1 | 1 | |
| 1549600 | 4667 | 4 | * | 56 | 2 | 1 | 3 | Preferred |
| | | | | | | | | candidate |
| 1872406 | 28545 | 5 | * | 56 | 7 | 2 | 1 | |
| 1893110 | 27610 | 4 | 48 | 57 | 5 | 2 | 0 | |
| 00757 | 26399 | 4 | 47 | 57.5 | 4 | 1 | 3 | Preferred |
| | | | | | | | | candidate |
| 75951 | 17615 | 5 | 48.4 | 58 | 8.5 | 2 | 1 | |
| 19799 | 28828 | 4 | 46.9 | 58 | 11 | 2 | 2 | Most |
| | | | | | | | | preferred |
| | | | | | | | | candidate |
| 1889560 | 34686 | 5 | 46 | 58 | 25 | 2 | 1 | |
| 1893791 | 29225 | 5 | 49 | 59 | 13 | 2 | 0 | |
| 1892710 | 15139 | 3 | 99 | 61 | 7 | 1 | 1 | |
| 26517 | 26612 | 6 | 52 | 65 | 15 | 1 | 1 | Out of |
| | | | | | | | | window |
| 65510 | 26651 | 5 | 346.2 | 96 | 10 | 0 | 0 | Out of |
| | | | | | | | | window |
| 9306 | 18685 | 4 | 200 | 150 | 14 | 0 | 0 | Out of |
| | | | | | | | | window |
| 24498 | 32275 | 6 | 493 | 409 | 3 | 0 | 0 | Out of |
| | | | | | | | | window |
| 88018 | 23923 | 5 | 44.8 | * | 12 | 2 | 1 | |
| 1926247 | 22585 | 2 | 47.3 | 57.5 | 10 | 1 | 1 | |
| 1929618 | 20160 | 3 | 46.9 | 57.4 | 2 | 1 | 2 | |
| 1934717 | 65001 | 6 | 47.3 | 57.8 | 50 | 2 | 2 | Preferred |
| | | | | | | | | candidate |

* Maps to different places on the chromosome

### Example 6: Rust Resistance Resistant Genes)

Rust Resistance Gene(s) are Identified as Follows:

1. Sanger sequencing all of the available susceptible mutants, the resistant parental lines (42 and 34), Ae. sharonensis and Galil plants in the region of SNPs in the following scaffolds: scaffold 19799; scaffold 00757; scaffold 1934717; scaffold 1549600; scaffold 20860. The Sanger sequencing is performed in order to compare the resistant and the susceptible lines and to validate the presence of SNPs. 2. Identifying coding sequence of potential resistance genes and validating the presence of the SNPs within or near coding sequence.

3. Cloning the potential gene.

4. Introducing the sequence of the potential resistance gene(s) by transformation into susceptible wheat cultivar and examining the transformed plant resistance to rust diseases, validating the gene(s) function. Transformation is performed according to the method described in Ishida et al. (2015, ibid). Susceptibility and resistance of the transformed plant is examined as described in the Materials and Methods” section and in Example 4 hereinabove.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-40. (canceled)

41. A wheat plant comprising in its genome a heterologous polynucleotide segment conferring or enhancing resistance of the wheat plant to leaf rust and stripe rust diseases, wherein the heterologous polynucleotide segment comprises at least one scaffold having a nucleic acid sequence at least 70% homologous to the nucleic acid sequence set forth in any one of SEQ ID NOs:1-31 or a part thereof.

42. The wheat plant of claim 41, wherein the heterologous polynucleotide segment comprises at least one of:
a. at least one scaffold having a nucleic acid sequence at least 70%, 75%, 80%, 85%, 90%, or 95% homologous to a nucleic acid sequence selected from the group consisting of SEQ ID NO:1, SEQ ID NO:3, SEQ ID NO:31, SEQ ID NO:23, SEQ ID NO:2 and any combination thereof; and
b. a nucleic acid marker designated 2-3HS2, wherein the 2-3HS2 marker is amplified by a pair of primers comprising a forward primer comprising the nucleic acid sequence set forth in SEQ ID NO:32 and a reveres primer comprising the nucleic acid sequence set forth in SEQ ID NO:33, wherein said 2-3HS2 marker comprises a nucleic acid sequence at least 90% homologous to the nucleic acid sequence set forth in SEQ ID NO:34.

43. The wheat plant of claim 41, wherein the heterologous polynucleotide segment comprises a fragment of chromosome 6Ssh of Aegilops sharonensis Accession TH548, seed of which have been deposited with NCIMB Ltd. as the International Depositary Authority under Accession No. NCIMB 43567.

44. The wheat plant of claim 43, wherein the fragment of the Ae. sharonensis chromosome 6Ssh comprises at least one scaffold or a part thereof selected from the group consisting of: scaffold 19799 having the nucleic acid sequence of SEQ ID NO:1 positioned around 58Mbp; scaffold 00757 having the nucleic acid sequence of SEQ ID NO:3 positioned around 57.5Mbp; Scaffold 1934717 having the nucleic acid sequence of SEQ ID NO:31 positioned around 57.7 Mbp; scaffold 1549600 having the nucleic acid sequence of SEQ ID NO:23 positioned around 56Mbp; scaffold 20860 having the nucleic acid sequence of SEQ ID NO:2 positioned around 44Mbp; scaffold 1540406 having the nucleic acid sequence of SEQ ID NO:4 positioned around 32Mbp; scaffold 1900261 having the nucleic acid sequence of SEQ ID NO:5 positioned around 33Mbp; scaffold 1933170 having the nucleic acid sequence of SEQ ID NO:6 positioned around 34Mbp; scaffold 1531163 having the nucleic acid sequence of SEQ ID NO:7 positioned around 35Mbp; scaffold 1927703 having the nucleic acid sequence of SEQ ID NO:8 positioned around 35Mbp; scaffold 1895355 having the nucleic acid sequence of SEQ ID NO:9 positioned around 38Mbp; scaffold 1931208 having the nucleic acid sequence of SEQ ID NO:10 positioned around 38Mbp; scaffold 1935984 having the nucleic acid sequence of SEQ ID NO:11 positioned around 39Mbp; scaffold 1913977 having the nucleic acid sequence of SEQ ID NO:12 positioned around 42Mbp; scaffold 1916860 having the nucleic acid sequence of SEQ ID NO:13 positioned around 43Mbp; scaffold 1891763 having the nucleic acid sequence of SEQ ID NO:14 positioned around 44Mbp; scaffold 1937448 having the nucleic acid sequence of SEQ ID NO:15 positioned around 44Mbp; scaffold 1896571 having the nucleic acid sequence of SEQ ID NO:16 positioned around 46Mbp; scaffold 1528020 having the nucleic acid sequence of SEQ ID NO:17 positioned around 47Mbp; scaffold 1935712 having the nucleic acid sequence of SEQ ID NO:18 positioned around 47Mbp; scaffold 1893110 having the nucleic acid sequence of SEQ ID NO:19 positioned around 48Mbp or around 57Mbp; scaffold 1898148 having the nucleic acid sequence of SEQ ID NO:20 positioned around 51Mbp; scaffold 1934541 having the nucleic acid sequence of SEQ ID NO:21 positioned around 51Mbp; scaffold 1538547 having the nucleic acid sequence of SEQ ID NO:22 positioned around 56Mbp; scaffold 1872406 having the nucleic acid sequence of SEQ ID NO:24 positioned around 56Mbp; scaffold 1889560 having the nucleic acid sequence of SEQ ID NO:25 positioned around 58Mbp; scaffold 75951 having the nucleic acid sequence of SEQ ID NO:26 positioned around 58Mbp; scaffold 1893791 having the nucleic acid sequence of SEQ ID NO:27 positioned around 59Mbp; scaffold 1892710 having the nucleic acid sequence of SEQ ID NO:28 positioned around 61Mbp; scaffold 1926247 having the nucleic acid sequence of SEQ ID NO:29 positioned around 57.5 Mbp; Scaffold 1929618 having the nucleic acid sequence of SEQ ID NO:30 positioned around 57.4 Mbp; and any combination thereof.

45. The wheat plant of claim 41, wherein the heterologous polynucleotide segment is devoid of the nucleic acid sequence set forth in SEQ ID NO:35, SEQ ID NO:36, or a combination thereof.

46. The wheat plant of claim 41, wherein the heterologous polynucleotide segment is located within chromosome 6B of said wheat plant.

47. The wheat plant of claim 41, wherein the heterologous polynucleotide segment is a nucleic acid construct further comprising at least one regulatory element.

48. The wheat plant of claim 46, said wheat plant is selected from the group consisting of a plant homozygous for chromosome 6B comprising the heterologous polynucleotide segment and a plant heterozygous for chromosome 6B comprising a native wheat chromosome 6B and chromosome 6B comprising the heterologous polynucleotide segment.

49. The wheat plant of claim 41, said wheat plant shows a phenotype of enhanced resistance or tolerance to leaf rust and stripe rust diseases compared to a corresponding plant not comprising within its genome the heterologous polynucleotide segment.

50. A nucleic acid construct comprising an isolated polynucleotide comprising at least one scaffold having a nucleic acid sequence at least 70% homologous to a nucleic acid sequence set forth in any one of SEQ ID NOs:1-31 or a part thereof and at least one regulatory element, wherein the construct, when introduced into a wheat plant, confers or enhances resistance of the wheat plant to leaf rust disease and strip rust disease.

51. The construct of claim 50, wherein the isolated polynucleotide comprises at least one of
a. a nucleic acid sequence at least 70%, 75%, 80%, 85%, 90%, or 95% 95% homologous to a nucleic acid sequence selected from the group consisting of SEQ ID NO:1, SEQ ID NO:3, SEQ ID NO:31, SEQ ID NO:23, SEQ ID NO:2 and any combination thereof; and
b. a nucleic acid marker designated 2-3HS2, wherein the 2-3HS2 marker is amplified by a pair of primers comprising a forward primer comprising the nucleic acid sequence set forth in SEQ ID NO:32 and a reveres primer comprising the nucleic acid sequence set forth in SEQ ID NO:33, wherein said 2-3HS2 marker comprises a nucleic acid sequence at least 90% homologous to the nucleic acid sequence set forth in SEQ ID NO:34.

52. The construct of claim 50, wherein the isolated polynucleotide comprises a fragment of chromosome 6S sh of Aegilops sharonensis Accession TH548, seed of which have been deposited with NCIMB Ltd. as the International Depositary Authority under Accession No. NCIMB 43567.

53. The construct of claim 50, wherein the polynucleotide segment is devoid of the nucleic acid sequence set forth in SEQ ID NO:35, SEQ ID NO:36, or a combination thereof.

54. A method for producing a wheat plant having enhanced resistance to leaf rust and strip rust diseases, the method comprises introducing into at least one cell of a wheat plant susceptible to the rust diseases a heterologous polynucleotide segment or a nucleic acid construct comprising same, wherein the heterologous polynucleotide segment comprises at least one scaffold having a nucleic acid sequence at least 70% homologous to the nucleic acid sequence set forth in any one of SEQ ID NOs:1-31, thereby producing a wheat plant having enhanced resistance to said rust diseases compared to a corresponding control plant.

55. The method of claim 54, wherein the heterologous polynucleotide segment or the construct comprising same is introduced by a method selected from the group consisting of transforming into at least one cell of the wheat plant said isolated polynucleotide or construct comprising same and subjecting at least one cell of the wheat plant to genome editing using artificially engineered nucleases.

56. The method of claim 54, wherein the heterologous polynucleotide segment forms part of chromosome 6Ssh of Ae. sharonensis Accession TH548, said method comprises crossing the Ae. sharonensis Accession TH548 with the wheat plant susceptible to the rust disease.

57. The method of claim 54, wherein the heterologous polynucleotide segment is introduced into chromosome 6B of the at least one cell of the susceptible wheat plant.

58. A method for identifying and selecting wheat plants having enhanced resistance to leaf rust and stripe resistance, comprising the steps of:
a. providing a plurality of wheat plants;
b. examining a nucleic acid sample obtained from each of the plurality of wheat plants for the presence of a heterologous polynucleotide segment comprising at least one scaffold having a nucleic acid sequence at least 70% homologous to a nucleic acid sequence set forth in any one of SEQ ID NOs:1-31 or parts thereof; optionally
c. examining a nucleic acid sample obtained from each of the plurality of wheat plant for the presence of a marker having the nucleic acid sequence set forth in SEQ ID NO:34; and
d. selecting wheat plants comprising the heterologous polynucleotide segment.

59. The method of claim 58, wherein the control plant is a wheat plant susceptible to the rust diseases.

60. The method of claim 59, wherein the control plant is of the same genetic background.
